SEQUENCE LISTING (4 sequences)

SEQ ID NO: 1 - protein, 29 aa, Homo sapiens
HSQGTFTSDYSKYLDSRRAQDFVQWLMNT

SEQ ID NO: 2 - protein, 30 aa, Homo sapiens
HAEGTFTSDVSSYLEGQAAKEFIAWLVKGR

SEQ ID NO: 3 - protein, 31 aa, Homo sapiens
HAEGTFTSDVSSYLEGQAAKEFIAWLVKGRG

SEQ ID NO: 4 - protein, 30 aa, Artificial Sequence
  chimeric GLP-1/glucagon peptide
HSQGTFTSDKSEYLDSERARDFVAWLEAGG

(19) United States
(12) Patent Application Publication
(10) Pub. No.: US 2022/0249617 A1
(43) Pub. Date: Aug. 11, 2022

(54) CYCLODEXTRIN BASED INJECTABLE COFORMULATIONS OF SGLT2 INHIBITORS AND INCRETIN PEPTIDES

(71) Applicant: MEDIMMUNE LIMITED, CAMBRIDGE (GB)

(72) Inventors: ANNE-LAURE LAINE, CAMBRIDGE (GB); LUTZ JERMUTUS, CAMBRIDGE (GB); ANA GOMES DOS SANTOS, CAMBRIDGE (GB)

(21) Appl. No.: 17/612,020

(22) PCT Filed: May 20, 2020
(86) PCT No.: PCT/EP2020/064128

§ 371 Date: Nov. 17, 2021

Related U.S. Application Data
(60) Provisional application No. 62/850,710, filed on May 21, 2019.

(51) Int. Cl. A61K 38/26; A61K 31/724; A61K 31/7034; A61K 45/06

(52) U.S. Cl. CPC A61K 38/26; A61K 45/06; A61K 31/7034; A61K 31/724

(57) ABSTRACT

Provided herein are coformulations comprising cyclodextrin that allow for concurrent, subcutaneous administration of sodium glucose co-transporter 2 inhibitors (SGLT2i), such as dapagliflozin, and incretin peptides, such as GLP-1/Glucagon dual agonist peptides.

The content of the electronically submitted sequence listing in ASCII text file (Name: GLPGG-202-US—PSP_SL.TXT; Size: 4,096 kilobytes; and Date of Creation: May 21, 2019) filed with the application is incorporated herein by reference in its entirety.

### BACKGROUND

A number of complex progressive diseases including asthma, cancer, and diabetes, to name a few, are driving world-wide disease burden. In order to adequately control the progression of these heterogeneous diseases, combination therapies have been demonstrated to be an efficacious medication strategy. Typically, patients start with a single drug to both control symptoms and stall disease progression, to which further drugs are added as the underlying pathophysiology worsens over time and symptoms become less controlled.

Type 2 diabetes (T2D) is a metabolic disorder characterised by high levels of blood glucose which, if poorly controlled, can lead to life-threatening health complications. Failing an initial intervention of diet and exercise alone, use of anti-diabetic drugs is initiated where patients start on metformin mono-therapy. As the disease progresses and blood glucose returns into the diabetic range, additional medications with different mechanisms of actions are added. Eventually, T2D patients are on dual or triple therapy either containing metformin or insulin as one of the active ingredients of their drug “cocktail.” This significant medication burden often leads to low compliance.

Non-adherence to diabetes therapy is a well-recognised challenge, and one of the main contributors for patients failing glycaemic control. Typically, more than half of patients on anti-diabetic therapy are inadequately controlled, defined as having an HbA1c level of greater than 7.5%. This is driven by a combination of both underlying disease progression and poor compliance. Based on clinical data from the United Kingdom, less than 15% of patients manage to adhere to their diabetes medications. Adherence rates correlate with the complexity of the regimen and decline from monotherapy to combination with the lowest adherence associated with the combinations of oral and injectable medications.

Two of the newest generations of anti-diabetic drugs, sodium glucose co-transporter 2 inhibitors (SGLT2is) and incretin agonists, are administered as oral and injectable medications, respectively. Accordingly, co-formulations that can significantly contribute to increased compliance by offering a convenient and simultaneous administration for two drugs that otherwise are required to be taken separately (e.g., one as an oral and another as an injectable) are needed.

### BRIEF SUMMARY OF THE INVENTION

Provided herein are pharmaceutical co-formulations comprising (i) incretin peptides, including, in particular, lipidated incretin peptides, (ii) sodium glucose co-transporter 2 inhibitors (SGLT2is), and (iii) cyclodextrins.

In one instance, a liquid pharmaceutical composition comprises (i) a lipidated incretin peptide, (ii) a sodium glucose co-transporter 2 inhibitor (SGLT2i), and (iii) a cyclodextrin.

In one instance, the incretin peptide is monolipidated. In one instance, the incretin peptide is a GLP-1/glucagon dual agonist peptide. In one instance, the incretin peptide is MEDIO382, liraglutide, or semaglutide.

In one instance, the SGLT2i is dapagliflozin.

In one instance, the cyclodextrin is a beta cyclodextrin. In one instance, the beta cyclodextrin is hydroxypropyl-β-cyclodextrin. In one instance, the cyclodextrin is sulfobutyl ether cyclodextrin.

In one instance, the lipidated incretin peptide is present at a concentration of about 0.5 mg/mL. In one instance, the SGLT2i is present in a concentration of about 17 mg/ml. In one instance, the cyclodextrin is present at a concentration of about 7% w/v.

In one instance, the SGLT2i and the cyclodextrin have a stoichiometry of about 1:1.

In one instance, the composition has a pH of about 6.5 to about 8. In one instance, the composition has a pH of about 7 to about 8. In one instance, the composition has a pH of about 7.

In one instance, the composition has a volume of 1 mL or less.

In one instance, the composition is for parenteral administration. In one instance, the parenteral administration is subcutaneous administration.

In one instance, the composition comprises inclusion complexes comprising the lipidated incretin peptide, the SGLT2i, and the cyclodextrin.

In one instance, the composition does not contain fibrils of the lipidated incretin peptide.

In one instance, the composition does not decrease the affinity of the lipidated incretin peptide for the GLP-1 receptor and/or the glucagon receptor.

In one instance, administration of the composition to a rat produces a lipidated incretin peptide Cmax of about 390 ng/ml, a lipidated incretin peptide T max of about 1 hour, a lipidated incretin peptide half-life of about 5 hours, and/or a lipidated incretin peptide AUC_{0-inf} of about 3500-4000 ng.hr/mL.

Also provided herein is an injection pen comprising any composition provided herein. In one instance, the injection pen delivers about 600 μL of the composition.

Also provided herein is a method of treating type 2 diabetes in a subject in need thereof comprising administering any composition provided herein to the subject. In one instance, the subject is overweight or obese.

Also provided herein is a method of treating Nonalcoholic Steatohepatitis (NASH) or Nonalcoholic Fatty Liver Disease (NAFLD) in a subject in need thereof comprising administering any composition provided herein to the subject. In one instance, the subject is overweight or obese.

Also provided herein is a method of reducing liver fat in a subject in need thereof comprising administering any composition provided herein to the subject. In one instance, the subject is overweight or obese.

In one instance of the methods, the administration delivers about 10 mg of the SGLT2i and/or about 300 μg of lipidated incretin peptide to the patient. In one instance, the administration is an adjunct to diet and exercise.

### BRIEF DESCRIPTION OF DRAWINGS

FIG. 1 shows the chemical structure, chemical formula (C₁₆₇H₂₅₂N₄₂O₅₅), and molecular weight (3728.09), for MEDI0382 (SEQ ID NO:4).

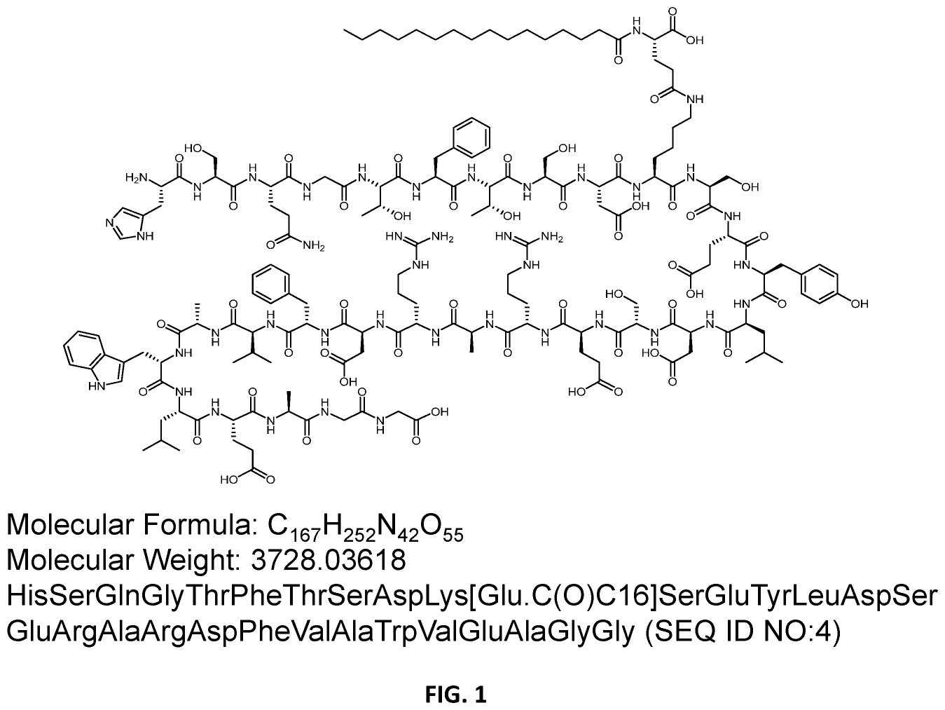

FIG. 2 provides a phase solubility diagram for dapagliflozin (DPZ):hydroxypropyl-β-cyclodextrin (HPβCD) complexes and empagliflozin (EPZ):HPβCD complexes. The apparent solubilities of DPZ and EPZ increase with increasing concentrations of HPβCD. (See Example 1.)

FIG. 3 shows the results of aggregation kinetic studies of MEDI0382 (SEQ ID NO:4) in various formulations including in buffer, in 7% HPβCD, and co-formulated with DPZ in 7% HPβCD. (A) Graph showing the time course of fibril formation followed by ThT fluorescence intensity measurement. The results show a full inhibition of fibrillation upon addition of cyclodextrin, both in presence and absence of DPZ. The data is presented as mean±SD (n=3). (B) Graph showing a characterization of the secondary structure of MEDI0382 in buffer and 7% HPβCD by far UV circular dichroism pre- and post-incubation at 37° C. MEDI0382 in buffer shows a typical alpha-helix profile at T0, and the apparition of β-Sheet structure at Tend (218 nm), confirming the presence of fibrils. When formulated in HPβCD, a shift of the CD spectrum was observed, but it remained unchanged over the incubation period. (See Example 2.)

FIG. 4 shows representative TEM images of MEDI0382 pre -and post-incubation at 37° C., confirming fibrils formation in the formulation of MEDI0382 in buffer only (scale bars=200 nm). (See Example 2.)

FIG. 5 shows representative TEM images of liraglutide in buffer and in HPβCD and associated vehicles post Tht assay confirming fibrils formation in the formulation of liraglutide in buffer only. (scale bars=200 nm) (See Example 2.)

FIG. 6 shows the liraglutide FTIR spectrum post Tht assay. (See Example 2.)

FIG. 7 shows the characterization of MEDIO382-HPβCD interaction. (A) Graph providing a qualitative evaluation of cyclodextrin cavity occupancy through ANS fluorescence measurement. Due to the properties of ANS to emit fluorescence upon complexation with HPβCD, ANS was used as a probe to assess the extent of free cavity in various formulations including in 7% HPβCD vehicle, MEDT0382 in 7% HPβCD, DPZ in 7% HPβCD and MEDT0382+DPZ in 7% HPβCD. The buffer vehicle and MEDI0382 in buffer were used as control. Lower fluorescence indicates higher occupancy. Data are represented as means±S.D. (n=3) (B) Graph showing intrinsic tryptophan (Trp) fluorescence measurements. Trp fluorescence emission spectrum informs on microenvironment change according to the formulation. MEDI0382+DPZ formulation was not measured due to interference from DPZ. Data are represented as means±S.D. (n=3). (See Examples 3 and 4.)

FIG. 8 shows a near UV circular dichroism spectra of MEDI0382 in buffer and in 7% HPβCD. The contribution from the aromatic residues are highlighted as follows: 285-310 nm for tryptophan (Trp), 275-285 nm for tyrosine (Tyr), and 255-270 nm for phenylalanine (Phe). (See Example 4.)

FIG. 9 shows typical ITC isotherms corresponding to the titrations of (A) HPβCD: DPZ and (B) HPβCD:MEDI0382. The titration of HPβCD: DPZ results in an exothermic profile whereas HPβCD:MEDI0382 gives an endothermic isotherm. (See Example 3.)

FIG. 10 shows region of 1H-1H NOESY spectra of MEDI0382 with 10% HPβCD (NMR water suppression). (A) NOESY regions focusing on the interactions between aromatic residue and HPβCD. (B) Schematic representation of the interaction with Trp. (C) NOESY regions focusing on the interactions between the palmitic lipid chain. (D) Schematic representation of the interaction. (See Examples 4 and 5.)

FIG. 11 shows (A) snapshots after 100 ns simulation of MEDI0382:HPβCD complex started from HPβCD docked onto the peptide. The lipid chain forms an inclusion complex with HPβCD. (B) Quantification of the types of interactions between HPβCD and the residues of the peptide are also shown. On average over the simulation specific hydrogen bonds (grey bars) are formed between HPβCD and side chain atoms and in many cases a water molecule (black bar) is bridging the interaction. (See Example 5.)

FIG. 12 shows Trp fluorescence (left) and characterization of MEDI0382 by far UV circular dichroism in the presence and absence of cyclodextrin at pH 6.5 and 8. (See Example 6.)

FIG. 13 shows the aggregation kinetic profile followed by Tht fluorescence of MEDI0382 in the presence and absence of cyclodextrin at pH 6.5 and 8. (See Example 6.)

FIG. 14 shows AFM and TEM images of MEDI0382 in buffer and in HPβCD post-Tht assay at pH 6.5 and pH 8.0. (See Example 6.)

FIG. 15 shows the far UV CD spectrum of MEDIO382 post-Tht assay in the presence and absence of cyclodextrin at pH 6.5 and 8. The secondary structure composition is indicated in the table (See Example 6.)

FIG. 16 shows the results of a MEDIO382 aggregation kinetics assay in the presence and absence of cyclodextrin at pH 6.5 and 8. (See Example 6.)

FIG. 17 shows the aggregation kinetic profile followed by Tht fluorescence of liraglutide in the presence and absence of cyclodextrin at pH 6.5 and 8. (See Example 6.)

FIG. 18 shows the far UV CD spectrum of liragludite post-Tht assay in the presence and absence of cyclodextrin at pH 6.5 and 8. (See Example 6.)

FIG. 19 shows the in vitro and in vivo performance of the co-formulation. In vitro potency assay on (A) GLP1 receptor (GLP1 R) and (B) Glucagon Receptor (GluR). Plasma concentration versus time profile of (C) MEDIO382 and (D) dapagliflozin after subcutaneous injection in rats. (See Example 7.)

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

### I. Definitions

As used in this specification, the singular forms “a,” “an” and “the” specifically also encompass the plural forms of the terms to which they refer, unless the content clearly dictates otherwise. As such, the terms “a” (or “an”), “one or more,” and “at least one” can be used interchangeably herein.

The term “about” is used herein to mean approximately, in the region of, roughly, or around. When the term “about” is used in conjunction with a numerical range, it modifies that range by extending the boundaries above and below the numerical values set forth. In general, unless otherwise stated, the term “about” is used herein to modify a numerical value above and below the stated value by a variance of 20%.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Technical and scientific terms used herein have the meaning commonly understood by one of ordinary skill in the art to which the present application pertains, unless otherwise defined. Reference is made herein to various methodologies and materials known to those of skill in the art. Standard reference works setting forth the general principles of peptide synthesis include W. C. Chan and P. D. White., “Fmoc Solid Phase Peptide Synthesis: A Practical Approach”, Oxford University Press, Oxford (2004). In addition, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The terms “peptide,” “polypeptide,” “protein,” and “protein fragment” are used interchangeably herein to refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The term “peptide” further includes peptides that have undergone post-translational or post-synthesis modifications, for example, glycosylation, acetylation, phosphorylation, amidation, derivatization by known protecting/blocking groups, proteolytic cleavage, or modification by non-naturally occurring amino acids. A “peptide” can be part of a fusion peptide comprising additional components such as, an Fc domain or an albumin domain, to increase half-life. A peptide as described herein can also be derivatized in a number of different ways.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

The term “isolated” refers to the state in which peptides or nucleic acids, will generally be in accordance with the present disclosure. Isolated peptides and isolated nucleic acids will be free or substantially free of material with which they are naturally associated such as other peptides or nucleic acids with which they are found in their natural environment, or the environment in which they are prepared (e.g. cell culture) when such preparation is by recombinant DNA technology practiced in vitro or in vivo. Peptides and nucleic acid can be formulated with diluents or adjuvants and still for practical purposes be isolated—for example the peptides will normally be mixed with gelatin or other carriers if used to coat microtitre plates for use in immunoassays, or will be mixed with pharmaceutically acceptable carriers or diluents when used in diagnosis or therapy.

A “recombinant” peptide refers to a peptide produced via recombinant DNA technology. Recombinantly produced peptides expressed in host cells are considered isolated for the purpose of the present disclosure, as are native or recombinant polypeptides which have been separated, fractionated, or partially or substantially purified by any suitable technique.

The terms “fragment,” “analog,” “derivative,” or “variant” when referring to an incretin peptide include any peptide which retains at least some desirable activity, e.g., binding to glucagon and/or GLP-1 receptors. Fragments of incretin peptides provided herein include proteolytic fragments, deletion fragments which exhibit desirable properties during expression, purification, and/or administration to a subject.

The term “variant,” as used herein, refers to a peptide that differs from the recited peptide due to amino acid substitutions, deletions, insertions, and/or modifications. Variants can be produced using art-known mutagenesis techniques. Variants can also, or alternatively, contain other modifications-for example a peptide can be conjugated or coupled, e.g., fused to a heterologous amino acid sequence or other moiety, e.g., for increasing half-life, solubility, or stability. Examples of moieties to be conjugated or coupled to a peptide provided herein include, but are not limited to, albumin, an immunoglobulin Fc region, polyethylene glycol (PEG), and the like. The peptide can also be conjugated or produced coupled to a linker or other sequence for ease of synthesis, purification or identification of the peptide (e.g., 6-His), or to enhance binding of the polypeptide to a solid support.

The term “pharmaceutical co-formulation” refer to compositions containing an incretin peptide and a SGLT2i along with e.g., pharmaceutically acceptable carriers, excipients, or diluents for administration to a subject in need of treatment, e.g., a human subject with type 2 diabetes.

The term “pharmaceutically acceptable” refers to compositions that are, within the scope of sound medical judgment, suitable for contact with the tissues of human beings and animals without excessive toxicity or other complications commensurate with a reasonable benefit/risk ratio.

The term “pharmaceutically acceptable carrier” refers to one or more non-toxic materials that do not interfere with the effectiveness of the biological activity of the incretin peptide and/or SGLT2i.

An “effective amount” is that amount of an incretin peptide and/or SGLT2i, the administration of which to a subject, either in a single dose or as part of a series, is effective for treatment, e.g., treatment of type 2 diabetes. This amount can be a fixed dose for all subjects being treated, or can vary depending upon the weight, health, and physical condition of the subject to be treated, the extent of weight loss or weight maintenance desired, and other relevant factors.

The term “subject” is meant any subject, particularly a mammalian subject, in need of treatment with a pharmaceutical co-formulation provided herein. Mammalian subjects include, but are not limited to, humans, dogs, cats, guinea pigs, rabbits, rats, mice, horses, cattle, bears, cows, apes, monkeys, orangutans, and chimpanzees, and so on. In one instance, the subject is a human subject.

As used herein, a “subject in need thereof” refers to an individual for whom it is desirable to treat, e.g., a subject with type 2 diabetes.

Terms such as “treating” or “treatment” or “to treat” refer to therapeutic measures that cure and/or halt progression of a diagnosed pathologic condition or disorder. Terms such as “preventing” refer to prophylactic or preventative measures that prevent and/or slow the development of a targeted pathologic condition or disorder. Thus, those in need of treatment include those already with the disease or condition. Those in need of prevention include those prone to have the disease or condition and those in whom the disease or condition is to be prevented. For example, the phrase “treating a patient” having type 2 diabetes refers to reducing the severity of the disease or condition to an extent that the subject no longer suffers discomfort and/or altered function due to it. Treating includes therapeutic measures that slow down or lessen the symptoms of a diagnosed pathologic condition or disorder.

As used herein a “GLP-1/glucagon agonist peptide” is a chimeric peptide that exhibits activity at the glucagon receptor of at least about 1%, 5%, 10%, 20%, 30%, 40%, 50%, 60%, 70%, 80%, 90%, 95%, or more relative to native glucagon and also exhibits activity at the GLP-1 receptor of about at least about 1%, 5%, 10%, 20%, 30%, 40%, 50%, 60%, 70%, 80%, 90%, 95%, or more relative to native GLP-1.

As used herein the term “native glucagon” refers to naturally-occurring glucagon, e.g., human glucagon, comprising the sequence of HSQGTFTSDYSKYLDSRRAQDFVQW LMNT (SEQ ID NO:1). The term “native GLP-1” refers to naturally-occurring GLP-1, e.g., human GLP-1, and is a generic term that encompasses, e.g., GLP-1(7-36) amide (HAEGT FTSDVSSYLEGQAAKEFIAWLVKGR; SEQ ID NO:2), GLP-1(7-37) acid (HAEGT FTSDVSSYLEGQAAKEFIAWLVKGRG; SEQ ID NO:3), or a mixture of those two compounds. As used herein, a general reference to “glucagon” or “GLP-1” in the absence of any further designation is intended to mean native human glucagon or native human GLP-1, respectively. Unless otherwise indicated, “glucagon” refers to human glucagon, and “GLP-1” refers to human GLP-1.

### II. Incretin Peptides

The pharmaceutical co-formulations provided herein comprise incretin peptides, including, in particular, lipidated incretin peptides. Incretin peptides are agonists of GLP-1, and they include approved GLP-1 mono-agonists as well as dual or triple agonists such as MEDI0382, a GLP-1/Glucagon receptor dual agonist. (See Henderson S J et al., Diabetes Obes Metab.18:1176-90 (2016), which is herein incorporated by reference in its entirety.) Lipidation can prolong the blood circulation of incretin peptides. In addition, as shown herein, aromatic residues in a lipid chain can interact with a cyclodextrin (e.g. HPβCD) in a fashion that decreases aggregation of the incretin peptide.

In one instance, the incretin peptide for use in the pharmaceutical co-formulations provided herein is MEDIO382. MEDI0382 is a 30 amino acid linear peptide with the sequence of HSQGTFTSDX₁₀SEYLDSERARDFVAWLEAGG-acid, wherein X₁₀=lysine with a palmitoyl group conjugated to the epsilon nitrogen, through a gamma glutamic acid linker (i.e., K(gE-palm)) (SEQ ID NO:4). MEDT0382 is palmitoylated to extend its half-life by association with serum albumin, thus reducing its propensity for renal clearance. MEDI0382 has been designed to elicit all the positive therapeutic attributes related to GLP-1 analogues (see Meier J J., Nat Rev Endocrinol. 8:728-42. (2012), which is herein incorporated by reference in its entirety) including effective glycemic control, gastric emptying delay, induction of satiety and reduction of body weight, coupled with the additional effect of glucagon on energy expenditure and metabolic rate. To extend the systemic circulation time of the peptide, a C16 chain was covalently attached to its amino acid sequence allowing reversible binding to serum albumin. This strategy was previously successfully applied to liraglutide, an approved GLP-1 peptide mono-agonist marketed under the trade name of Victoza®. During preclinical studies, repeat injections of MEDI0382 led to marked weight loss and robust glucose control in DIO mice and non-human primates. Currently under clinical evaluation for the treatment of overweight or obese patients with type 2 diabetes, MEDI0382 has shown glucose, weight and liver fat lowering efficacy in overweight and obese patients with type 2 diabetes. (See Ambery P, et al., Lancet. 391:2607-18 (2018), which is herein incorporated by reference in its entirety.)

In one instance, the incretin peptide is MEDIO382, semaglutide, or liraglutide.

Additional incretin peptides can also be used in the pharmaceutical co-formulations provided herein. Exemplary lipidated incretin peptides are provided, for example, in Wang et al., J. Control Release 241:25-33 (2016), which is herein incorporated by reference. In certain instances, a lipidated incretin peptide for use in a pharmaceutical co-formulation provided herein is a mono-lipidated incretin peptide.

Incretin peptides for use in the pharmaceutical co-formulations provided herein can be acylated.

Incretin peptides for use in the pharmaceutical co-formulations provided herein can be associated with a heterologous moiety, e.g., to extend half-life. The heterologous moiety can be a protein, a peptide, a protein domain, a linker, an organic polymer, an inorganic polymer, a polyethylene glycol (PEG), biotin, an albumin, a human serum albumin (HSA), a HSA FcRn binding portion, an albumin binding domain, an enzyme, a ligand, a receptor, a binding peptide, a non-FnIII scaffold, an epitope tag, a recombinant polypeptide polymer, and a combination of two or more of such moieties.

Incretin peptides can be made by any suitable method. For example, in certain embodiments the incretin peptides are chemically synthesized by methods well known to those of ordinary skill in the art, e.g., by solid phase synthesis as described by Merrifield (1963, J. Am. Chem. Soc. 85:2149-2154). Solid phase peptide synthesis can be accomplished, e.g., by using automated synthesizers, using standard reagents, e.g., as explained in Example 1 of WO 2014/091316.

Alternatively, incretin peptides can be produced recombinantly using a convenient vector/host cell combination as would be well known to the person of ordinary skill in the art. A variety of methods are available for recombinantly producing incretin peptides. Generally, a polynucleotide sequence encoding the incretin peptide is inserted into an appropriate expression vehicle, e.g., a vector which contains the necessary elements for the transcription and translation of the inserted coding sequence. The nucleic acid encoding the incretin peptide is inserted into the vector in proper reading frame. The expression vector is then transfected into a suitable host cell which will express the incretin peptide. Suitable host cells include without limitation bacteria, yeast, or mammalian cells. A variety of commercially-available host-expression vector systems can be utilized to express incretin peptides.

### III. Co-Formulations

Provided herein are co-formulations comprising an incretin peptide (as discussed above), a sodium glucose co-transporter 2 inhibitor (SGLT2i), and a cyclodextrin.

SGLT2is are a class of medicines for use with diet and exercise to lower blood sugar in adults with type 2 diabetes. SGLT2is reduce blood glucose by blocking glucose reabsorption from the kidney. Since this mechanism is independent from insulin and is directly correlated to the level of blood glucose, SGLT2i offer a durable glucose-lowering approach that also minimizes hypoglycemic episodes.

Exemplary SGLT2is include dapagliflozin (DPZ), empagliflozin (EPZ), and canagliflozin. In certain instances, the SGLT2i is DPZ or EPZ. In certain instances, the SGLT2i is DPZ.

In certain instances, an SGLT2i (e.g., DPZ) is present in a pharmaceutical co-formulation provided herein at a concentration of about 17 mg/ml.

Cyclodextrins are cyclic oligosaccharides containing glucopyranose units. Cyclodextrins include alpha, beta, and gamma cyclodextrins, which have varying numbers of glucopyranose units. In certain instances, the cyclodextrin is a beta cyclodextrin. An exemplary cyclodextrin is hydroxypropyl-β-cyclodextrin (HPβCD). An additional exemplary cyclodextrin is sulfobutyl ether cyclodextrin.

In certain instances, a cyclodextrin (e.g., HPβCD) is present in a pharmaceutical co-formulation provided herein at a concentration of about 7% w/v.

In certain instances of the pharmaceutical co-formulations provided herein, the SGLT2i (e.g., DPZ) and the cyclodextrin (e.g., HPβCD) have a stoichiometry of about 1:1.

The pharmaceutical co-formulations provided herein can have a concentration of about 0.5 mg/mL of a lipidated incretin peptide (e.g. MEDIO382).

As demonstrated herein, the incretin peptide (e.g. MEDI0382), SGLT2i (e.g., DPZ) and the cyclodextrin (e.g., HPβCD) can be present in inclusion complexes in the pharmaceutical co-formulations provided herein.

The pharmaceutical co-formulations provided herein can have a pH of at least 6.5. The pharmaceutical co-formulations provided herein can have a pH of at least 7.

The pharmaceutical co-formulations provided herein can have a pH of about 6.5 to about 8. The pharmaceutical co-formulations provided herein can have a pH of about 7 to about 8. The pharmaceutical co-formulations provided herein can have a pH of about 7.

The co-formulations can be for parenteral, e.g., subcutaneous, delivery. The co-formulations can be, for example, for delivery via a pen device. Accordingly, also provided herein are pens for injection comprising a pharmaceutical co-formulation provided herein.

In the co-formulations, the SGLT2i and the incretin peptide can share the same volume of injection. Pain and tolerability issues can arise with large volumes. Accordingly, the co-formulation can have a volume of 1 mL or less. A co-formulation can therefore be designed to be administered in a volume of about 600 μL. As demonstrated herein, a cyclodextrin (e.g., hydroxypropyl-β-cyclodextrin (HPβCD)) can be used as a solubility enhancer to accommodate a therapeutically effective dose of a SGLT2i in the required volume.

Incretin peptides are notoriously difficult to formulate due to their innate properties to self-associate and aggregate as well as their pH dependent solubility and stability. As demonstrated herein, a cyclodextrin (e.g., HPβCD) can be used to prevent aggregation of the incretin peptide. Accordingly, a composition provided herein can lack fibrils of the incretin peptide (e.g., MEDIO382). The presence of fibrils can be assessed, for example, using transmission electron microscopy (TEM) or a thioflavin T (ThT) assay (e.g., as demonstrated herein in Example 2).

As demonstrated herein, the presence of cyclodextrin and/or the SGLT2i in a co-formulation with an incretin peptide does not diminish the potency of the incretin peptide (e.g., MEDI0382). The potency of an incretin peptide (e.g., MEDI0382) can be assessed, for example, using in vitro and/or in vivo assays. For instance, the activity of an incretin peptide (e.g., MEDIO382) can be assessed based on its activity on GLP-1 and/or glucagon receptors (e.g., as measured by an EC50 in a cAMP accumulation assay, optionally as demonstrated herein in Example 7.)

### IV. Methods of Treating

This disclosure provides a method of treating type 2 diabetes, comprising administering to a subject in need of treatment a pharmaceutical co-formulation provided herein comprising a lipidated incretin peptide (e.g., MEDI0382) and a SGLT2i (e.g., DPZ). In certain instances, the administration is an adjunct to diet and exercise. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese.

This disclosure provides a method of reducing liver fat comprising administering to a subject in need of treatment a pharmaceutical co-formulation provided herein comprising a lipidated incretin peptide (e.g., MEDIO382) and a SGLT2i (e.g., DPZ). The reduction of liver fat can lead to enhanced insulin sensitivity and/or improved liver function. In certain instances, the administration reduces hemoglobin A1c (HbA1c) levels. In certain instances, the administration is an adjunct to diet and exercise. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese. In certain instances, the subject has type 2 diabetes mellitus.

This disclosure provides a method of treating Nonalcoholic Steatohepatitis (NASH) comprising administering to a subject in need of treatment a pharmaceutical co-formulation provided herein comprising a lipidated incretin peptide (e.g., MEDIO382) and a SGLT2i (e.g., DPZ). In certain instances, the administration is an adjunct to diet and exercise. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese. In certain instances, the subject has type 2 diabetes mellitus.

This disclosure provides a method of treating Nonalcoholic Fatty Liver Disease (NAFLD) comprising administering to a subject in need of treatment a pharmaceutical co-formulation provided herein comprising a lipidated incretin peptide (e.g., MEDIO382) and a SGLT2i (e.g., DPZ). In certain instances, the administration is an adjunct to diet and exercise. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese. In certain instances, the subject has type 2 diabetes mellitus.

This disclosure provides a method of treating obesity or an obesity-related disease or disorder, of reducing body weight, of reducing body fat, of preventing weight gain, of preventing fat gain, of promoting weight loss, of promoting fat loss, of treating a disease or condition caused or characterized by excess body weight or excess body fat, of managing weight, of improving glycemic control, or of achieving glycemic control wherein the method comprises administering to a subject in need of treatment a pharmaceutical co-formulation provided herein comprising a lipidated incretin peptide (e.g., MEDI0382) and a SGLT2i (e.g., DPZ). In certain instances, the administration is an adjunct to diet and exercise. In certain instances, the subject has a BMI of 27 to 40 kg/m². In certain instances, the subject has a BMI of 30 to 39.9 kg/m². In certain instances, the subject has a BMI of at least 40. In certain instances, the subject is overweight. In certain instances, the subject is obese. In certain instances, the subject has type 2 diabetes mellitus.

Examples of other obesity-related (excess body weight-related) disorders include without limitation: insulin resistance, glucose intolerance, pre-diabetes, increased fasting glucose, type 2 diabetes, hypertension, dyslipidemia (or a combination of these metabolic risk factors), glucagonomas, cardiovascular diseases such as congestive heart failure, atherosclerois, arteriosclerosis, coronary heart disease, or peripheral artery disease, stroke, respiratory dysfunction, or renal disease.

In certain instances, the route of administration of a pharmaceutical co-formulation provided herein comprising a lipidated incretin peptide (e.g., MEDI0382) and a SGLT2i (e.g., DPZ)is parenteral. In certain instances, the route of administration of a pharmaceutical co-formulation provided herein comprising a lipidated incretin peptide (e.g., MEDIO382) and a SGLT2i (e.g., DPZ) is subcutaneous. In certain instances, a pharmaceutical co-formulation provided herein comprising a lipidated incretin peptide (e.g., MEDI0382) and a SGLT2i (e.g., DPZ) is administered by injection, e.g., from a pen. In certain instances, a pharmaceutical co-formulation provided herein comprising a lipidated incretin peptide (e.g., MEDIO382) and a SGLT2i (e.g., DPZ) is administered by subcutaneous injection.

In certain instances, a pharmaceutical co-formulation provided herein comprising a lipidated incretin peptide (e.g., MEDI0382) and a SGLT2i (e.g., DPZ) can be administered once per day. In certain instances, a pharmaceutical co-formulation provided herein comprising a lipidated incretin peptide (e.g., MEDI0382) and a SGLT2i (e.g., DPZ) can be administered once per day via injection (e.g., subcutaneous administration). In certain instances, a pharmaceutical co-formulation provided herein comprising a lipidated incretin peptide (e.g., MEDI0382) and a SGLT2i (e.g., DPZ) can be administered once per day via injection (e.g., subcutaneous administration) over a period of at least one week, over a period of at least two weeks, over a period of at least three weeks, or over a period of at least four weeks.

### EXAMPLES

### Materials

HPLC water and acetonitrile were purchased from VWR (VWR Radnor, Pa., USA). Dapagliflozin was provided by AstraZeneca. Kleptose® HPB (2-hydroxypropyl-β-cyclodextrin) was provided by Roquette (Roquette Freres, Lestrem, France). Captisol® (sulfobutylether-β-cyclodextrin) was provided by Ligand pharmaceuticals (Ligand Pharmaceuticals, San Diego, Calif., USA. 8-anilino-1-naphthalenesulfonic acid (ANS) and thioflavin t (ThT) were purchased from Sigma-Aldrich (Sigma-Aldrich, St. Louis, Mo., USA). Sodium phosphate dibasic heptahydrate and sodium phosphate monobasic monohydrate were provided by J. T. Baker (J. T. Baker chemical co, Phillipsburg, N.J., USA).

### Solubility Screen

Dapagliflozin (DPZ) was weighed in a glass vial. The appropriate aqueous vehicle was added onto the powder to achieve a final concentration of 17 mg/mL, vortex mixed, and sonicated. The pass/fail criteria for the formulation was determined through visual observation.

### DPZ Phase Solubility in HPβCD (Kleptose HPB)

Various HPβCD (Kleptose HPB, Roquette) solutions at increasing concentrations were prepared in water ranging from 5% to 20% (w/v). Briefly, HPβCD was weighed in a volumetric flask and purified water was added up to 80% (v/v) of final volume. The flask was mixed until full solubilisation and made up to final volume with purified water. Approximately 30 mg of DPZ was weighed in each HPLC glass vial to which 500 μL of an appropriate HPβCD solution was added with a magnetic flea. Each concentration was done in duplicate. The formulations were left under magnetic stirring for 21 h 40 min. Each sample was then transferred to a 1.5 mL eppendorf and centrifuged at 13,000 rpm for 10 min. Then 200 μL was taken from the supernatant and centrifuged again in a 1.5 mL eppendorf for 30 min at 13,000 rpm. The samples were finally diluted in buffer A (95% HPLC water/5% ACN+0.03% TFA), and the concentration was measured by UPLC against a calibration curve validated with quality controls.

### Isothermal Titration Calorimetry

Isothermal titration calorimetry (ITC) measurements were carried out at 25° C. by titration of the cyclodextrin into a peptide or DPZ solution using the Microcal Auto ITC 200 (Malvern). MEDI0382 and DPZ solutions were prepared at 0.13 and 0.12 mM respectively, and the cyclodextrin (HPβCD) was prepared at 3 mM in the matching buffer. The experiments were performed in triplicate, and each run included 20 injections of 2 μL (first injection only 0.4 μL) with the stirring speed set at 750 rpm. The isotherms were fitted using the one set of binding sites model through Malvern Origin software.

### Tryptophan Fluorescence

Fluorescence measurements were performed on a F-7000 FL Spectrophotometer at room temperature. 100 μL of peptide formulation was added into a 96-well plate (half area) in triplicate. The excitation wavelength was set at 277 nm for selective excitation of tryptophan fluorescence. The fluorescence emission spectra were scanned between 285 and 385 nm. Both excitation and emission slits were set at 2.5 nm. Each spectrum was an average of three scans.

### Circular Dichroism (CD)

Circular dichroism spectra of freshly prepared peptide solution in 20 mM sodium phosphate (NaP) buffer pH 7.0 or in 7% HPβCD/20 mM NaP buffer pH 7.0 at 0.5 mg/mL were acquired at room temperature on a Jasco J-815 spectropolarimeter. The far-UV CD data were collected from 180 to 260 nm using a 0.1 mm path length cuvette, and the spectra were deconvoluted with the CONTINLL, SELCON3, and CDSSTR algorithms using CDPro software. The near-UV CD data were collected from 250 to 350 nm using a 1 cm path length cuvette.

### Kinetics of Aggregation

For aggregation kinetics experiments, MEDI0382 was monitored through the thioflavin T (ThT) binding assay and compared in the presence and in the absence of cyclodextrin. Fluorescence measurements were carried out on a Fluostar Optima Microplate Reader (BMG Labtech, Offenburg, Germany), which was thermostatted at 37° C. ThT binding to fibrils was monitored by using an excitation filter at 440 nm and recording the emission fluorescence at 480 nm. The formulations tested were 20 mM NaP buffer pH 7.0 with and without cyclodextrin at 7% w/v. MEDI0382 was formulated at 0.5 mg/mL and DPZ at 17 mg/mL. 100 1 μl of formulation were pipetted into the wells of a 96-well half area plate made of black polystyrene with a clear bottom (Coming 3881, US) to which 10 UL of a 0.5 mM ThT solution in water was added. Each sample was prepared in triplicate. A sealing tape and a sealing foil (Costar Thermowell) were used to prevent evaporation. Bottom reading of the plate was performed every 30 min with 5 min of shaking prior to each measurement. Each cycle was executed with the orbital shaker at 350 rpm, 5 flashes per well.

### NMR

2D NOESY NMR spectra were acquired with water suppression from solutions of MEDI0382 at 4.3 mg/ml in NaP buffer pH 7.6 with and without 10% HPβCD. All NMR experiments were run at temperature of 300 K on a 600 MHz Bruker Avance-III HD NMR spectrometer equipped with a 5 mm TCI cryoprobe (Bruker-Biospin) using standard pulse sequences from the Bruker library (TopSpin 3.5). Phase-sensitive NOESY experiments (pulse program “noesyesgpph”) were acquired using the excitation sculpting method for solvent suppression (Hwang T. L SAJ. Journal of Magnetic Resonance, Series A.]12(2):275-9 (1995)). Spectra were acquired with a relaxation delay of 1.5 s using a 4 K×512 data points over a spectral width 10 ppm in States-TPPI mode (Dominique Marion Mk, et al., Journal of Magnetic Resonance 85(2):393-9 (1989)) with acquisition times of 0.341 and 0.043 s in F2 and F1, respectively (zero-filling to 1K in F1). 128 scans and 16 dummy scans were collected for each F1 increment with a mixing time of 0.15 s. The data was processed using Topspin 3.5 software (Bruker-Biospin) with a sine-bell squared window function applied prior to Fourier transformation in both the F1 and F2 dimensions.

### Transmission Electron Microscopy (TEM)

MEDI0382 formulations pre- and post-incubation at 37° C. were adsorbed onto 400 mesh cupper/carbon film grids (EM resolutions), twice washed with deionised water, and subsequently negatively stained using 1.5% uranyl acetate in deionised water. Samples were viewed in a FEI Tecnai G2 electron microscope (Thermo Fisher Scientific) run at 120 keV using a 20 μm objective aperture to improve contrast. Images were taken using an AMT camera.

### Molecular Modeling

Molecular dynamics (MD) simulations was performed with the Desmond software (Proceedings of the ACM/IEEE Conference on Supercomputing (SC06), Tampa, Fla., Nov. 11-17, 2006). The initial geometry was generated as described below for each individual case. MEDI0382 peptide was build using the x-ray structure of a glucagon analogue, PDB code1BHO (Sturm NS, et al., J Med Chem. 41(15):2693-700 (1998)). The amino acids were manually mutated using the peptide editing tools in Maestro (Schr6dinger Release 2018-1: Jaguar, Schr6dinger, LLC, New York, N.Y., 2018). The missing amino acids towards the C-terminus was built without template. The side chain of Tyr10 was removed and replaced with K(γE-palm)C-16 fatty acid model manually. All carboxylic acids were kept charged except for the C-terminus that was protonated. The final 3D model of MEDI0382 was allowed to relax in an NPT molecular dynamics (MD) simulation of 10 ns. The equilibrated model was used as starting point for all further simulations. The 3D model of HPβCD was built from the x-ray structure of β-cyclodextrin (β-CD) extracted from the CSD data base (BCDEXD10) (Klaus Lindner WS, Carbohydrate Research.99(2):103-15 (1982). Four groups of 2-hydroxypropyl were manually added to the original β-CD structure. This geometry was relaxed to the nearest energy minimum. The relaxed 3D model of HPβCD was used as starting geometry for all further studies.

The peptide homology models were inserted in a system containing 50000 TIP3 models of water molecules with a 100×100×100A simulation box. The system was neutralized by adding Na⁺ ions. The peptide concentration was set at 0.55 mM, and the sodium concentration was set at 2.76 mM. All simulations were done using the OPLS3 force filed (OPLS3e, Schrödinger, Inc., New York, N.Y., 2013) (Shivakumar D, et al., J Chem Theory Comput. 8(8):2553-8 (2012)) for the peptide, cyclodextrin and Na ions. The initial system was allowed to go through a relaxation sequence of a) 100 ps Brownin Dynamics NVT T=10 K; b) 12 ps NVT MD T=10 K restraints on solute heavy atoms small time step; c) 12 ps NPT MD T=10 K restraints on solute heavy atoms; d) 12 ps NPT MD T=300 K restraints on solute heavey atoms; and e) NPT MD T=300 K no restraints. After the relaxation protocol, the production simulation was started using the NPT ensemble invoking the Nose-Hoover chian thermostat with a relaxation time of 1 ps to keep the temperature to 300K and the Martyna-Tobias-Klein barostat to keep the pressure to 1 atmosphere with a relaxation time of 2 ps. The RESPA algorithm was used to integrate the equation of motions with a time step of 2 fs.

### In Vitro Potency Assay

CHO-K1 cell lines stably expressing either GLP-1 or GCG receptors were stably transduced with a cAMP response element linked to a luciferase reporter gene to determine in vitro agonist potencies of MEDI0382 in buffer, in cyclodextrin, and co-formulated with DPZ. Briefly, cells were plated at 20,000 cells per well in 96-well white microtiter plates (Corning, USA) and incubated with serially diluted peptide samples for 4 hours prior to lysis and measurement of cAMP dependent luciferase activity using Steady-Glo luciferase substrate (Promega, USA). Plates were read on a SpectraMax Paradigm plate reader (Molecular Devices, USA), and 10-point concentration-response curves were generated in triplicate. Results were expressed as the relative potency of the test sample compared to a reference ligand by calculating the ratio of the reference and sample EC50 values from a 4-PL fit following a test for parallelism using SoftMax Pro software (Molecular Devices, USA), and the reported data was the mean of two independent assays. Curves were fitted using nonlinear regression analysis in GraphPad Prism software 6.03 (GraphPad, USA).

### MEDI0382 and DPZ Formulations for the PK Study

For the PK study, MEDI0382 alone in buffer was prepared at 0.5 mg/mL in 50 mM Na Phosphate buffer pH 7.8+1.85% propylene glycol (PG) (J. T. Baker). This buffer allows for comparison the PK profile to historical data.

The cyclodextrin vehicle used for the PK study was 7% w/v HPβCD in 50 mM Na Phosphate buffer pH 7.8+0.5% v PG. The PG was added to adjust the osmolarity of the formulations to 260 mOsm. Briefly, DPZ was solubilised in (7% w/v HPβCD in 50 mM NaP buffer pH 7.8+0.5% v/v PG) vehicle at a concentration of 5 mg/mL MEDI0382 was then added to achieve a concentration of 0.5 mg/mL. In parallel, MEDT0382 alone in buffer was prepared at 0.5 mg/mL in 50 mM NaP buffer pH 7.8+1.85% v/v PG. The formulations were then diluted to 1/10 with their corresponding vehicle.

The dose for the PK study were set at 0.5 mg/kg and 0.05 mg/kg for DPZ and MEDI0382 with a dose volume of 1 mL/kg.

Three animals per group were included, and serial blood sampling for PK evaluation occurred at 0.5, 1, 2, 4, 7, and 24 hr post dose. Both MEDI0382 and DPZ were analysed from plasma samples using validated methods consisting of plasma protein crash sample preparation followed by LC-MS/MS.

### Example 1: Cyclodextrin Increases the Solubility of Sglt2I

The recommended dose of DPZ is a 10 mg tablet once daily for monotherapy and add-on combination therapy with other glucose-lowering medications (recommendation from the European Medicines Agency) (EMA). Considering that DPZ is well absorbed after oral administration (reaching 78% absolute bioavailability) and that similar exposure can be expected from subcutaneous injection, the dose of DPZ for the co-formulation was fixed at 10 mg. The screening assay was therefore designed to target a concentration of 17 mg/mL, corresponding to a 10 mg dose in 600 μL dose volume. The excipients were selected based on several criteria such as, approval status for subcutaneous dosing, compatibility with the peptide, and/or precedence for increasing DPZ solubility. The excipients screened included PEG 400, PG, DSPE-PEG 2000, Glycerol, Kolliphor 188, HPβCD, and BSA. Most of the excipients were not able to achieve the required concentrations or to maintain DPZ in solution. Only the formulation containing cyclodextrin was successful and therefore taken forward for further evaluation as a potential co-formulation vehicle.

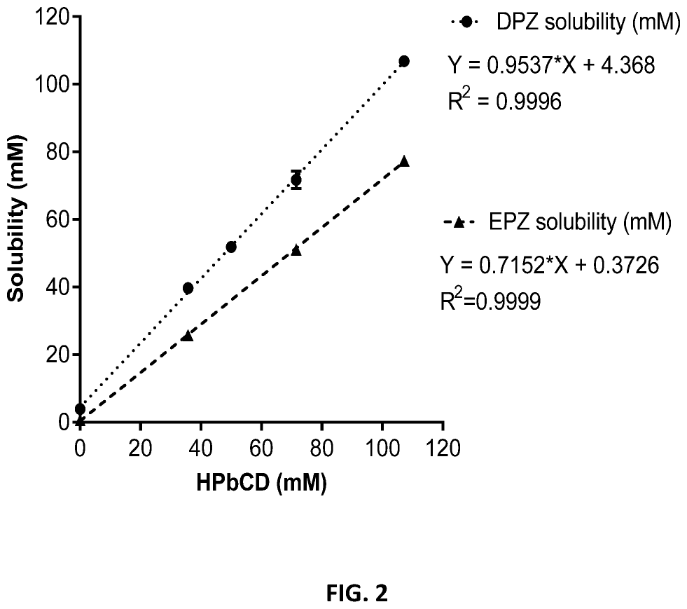

To get a better understanding of the enhancing capacity of cyclodextrin, a phase solubility study of DPZ in HPβCD was carried out (FIG. 2). The aqueous solubility of DPZ was experimentally measured at 1.6 mg/mL. Upon addition of HPβCD, DPZ solubility linearly increased with increasing HPβCD concentration, indicating the formation of inclusion complex of stoichiometry 1:1. The binding constant determined from the linear regression was 4.7×10³M⁻¹. Based on the phase solubility diagram, the amount of HPβCD required to increase the solubility up to 17 mg/mL is 5.5% (w/v). However, for solution formulation, it is recommended to not exceed 80% of saturation solubility to ensure stability, which set the level at 7% w/v. In order to confirm that HPβCD can be applied to other SGLT2i formulations, a phase solubility diagram was performed with empagliflozin (EPZ), which also showed linear solubility increase in presence of HPβCD (FIG. 2).

### Example 2: HpβCd Inhibits Medio382 Aggregation and Induces Conformational Change

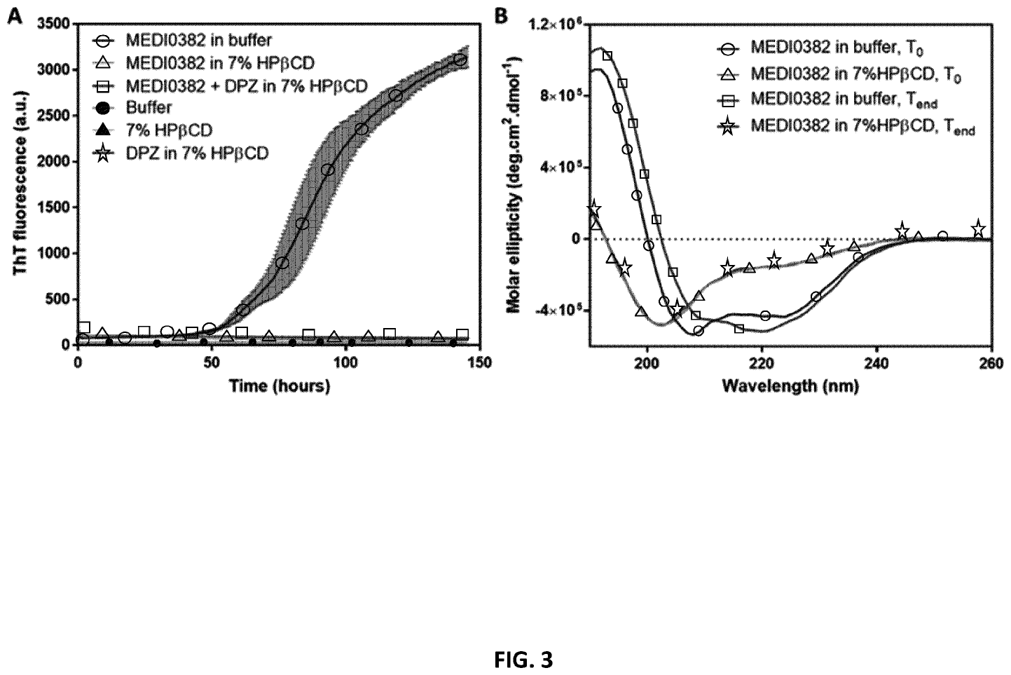

The tendency of peptides such as MEDIO382 to aggregate is one of the main issues in peptide formulation development. To assess the physical stability of MEDIO382 in a co-formulation, an aggregation kinetic study was performed using the thioflavin T (ThT) assay, which relies on the property of the ThT dye to emit highly enhanced fluorescence upon binding to fibrils (Biancalana M, et al., Biochim Biophys Acta.1804(7):1405-12 (2010)). The ThT assay was used to compare the aggregation of MEDIO382 at 37° C. in various formulation conditions, including with and without cyclodextrin and in the absence or presence of DPZ (FIG. 3A). The concentration of MEDIO382 was set at 0.5 mg/mL, which corresponds to a clinical dose of 300 μg.

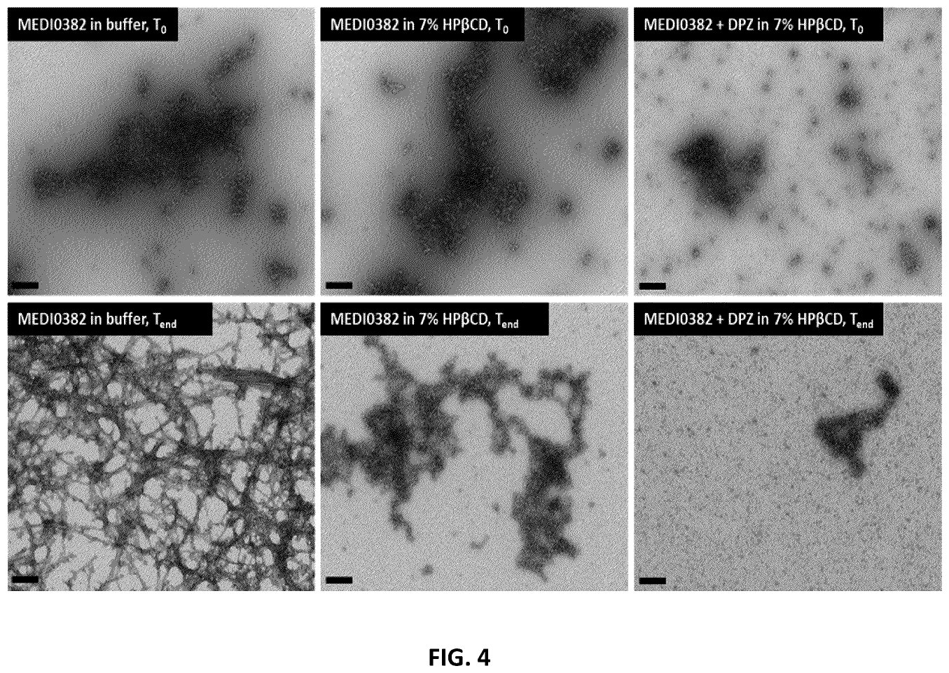

Prior to performing the aggregation test, the secondary structure of the freshly prepared formulations were analysed by far UV CD (FIG. 3B), and TEM pictures were acquired (FIG. 4). MEDIO382 solution in buffer showed a CD spectrum characteristic of α-helix structure, with a positive band at 192 nm and 2 negative bands at 207 and 222 nm (FIG. 3B). The deconvolution of the spectrum using CDpro confirmed the presence of a majority of α-helix conformation (51%) and a low proportion of j-sheet structure (11%). (See Table 1, below.) Upon formulation into HPβCD, a dramatic structural modification was observed with a positive band at 190 nm of lower intensity than that in the buffer, a negative band at 203 nm, and a low intensity band at 224 nm. The determination of the secondary structure using CDPro suggested a loss of helicity down to 18% compensated by an increase in 3-sheet and random coil. (See Table 1, below.)

TABLE 1
MEDI0382 CDPro

| | Alpha | Beta | | |
|---|---|---|---|---|
| | Helix | Sheet | Turn | Unordered |
| MEDI0382 in buffer, T₀ | 51.4% | 10.7% | 15.3% | 23.1% |
| MEDI0382 in 7% HPβCD, | 18.1% | 26.3% | 22.2% | 34.0% |
| T₀ | | | | |
| MEDI0382 in buffer, T_{end} | 49.2% | 12.8% | 13.5% | 24.7% |
| MEDI0382 in 7% HPβCD, | 18.1% | 26.7% | 21.7% | 34.8% |
| T_{end} | | | | |

No CD spectrum could be acquired for the co-formulation due to DPZ which possesses a chiral center. In all three MEDI0382 formulations, the absence of fibrils was evidenced by TEM pictures (FIG. 4).

The aggregation kinetic of MEDI0382 in buffer, in cyclodextrin, and co-formulated with DPZ in HPβCD was then monitored by ThT fluorescence measurement (FIG. 3A). The ThT profile of MEDI0382 in buffer followed a sigmoid curve indicative of fibril formation with an initial lag phase of 50 hr and a subsequent elongation phase which seemed to reach a plateau towards the end of the assay. TEM pictures taken at the final time point confirmed the presence of fibrils (FIG. 4). The far UV CD spectrum post-ThT assay indicated an increase in 3-sheet content as expected for fibrils, but it also showed a reasonably high percentage of helicity informing on the structure of the fibrils.

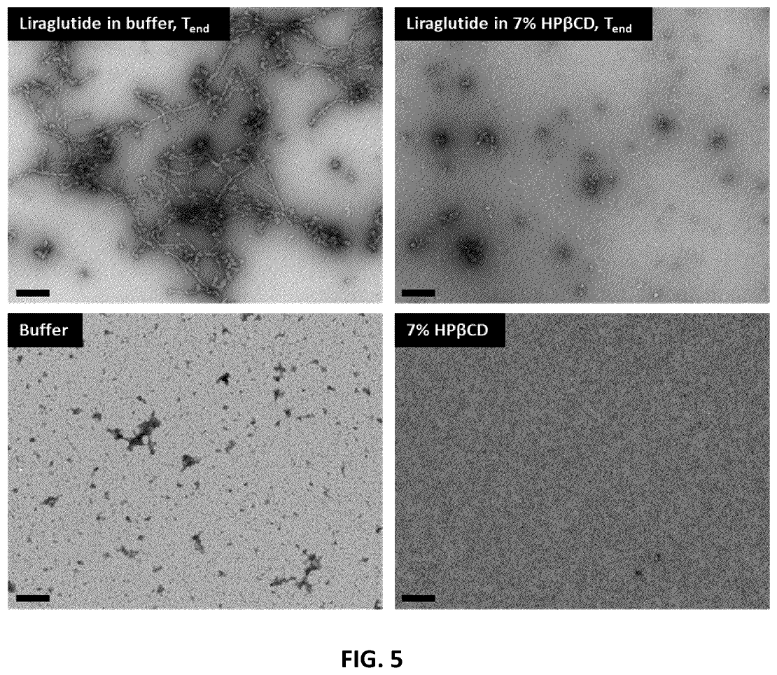

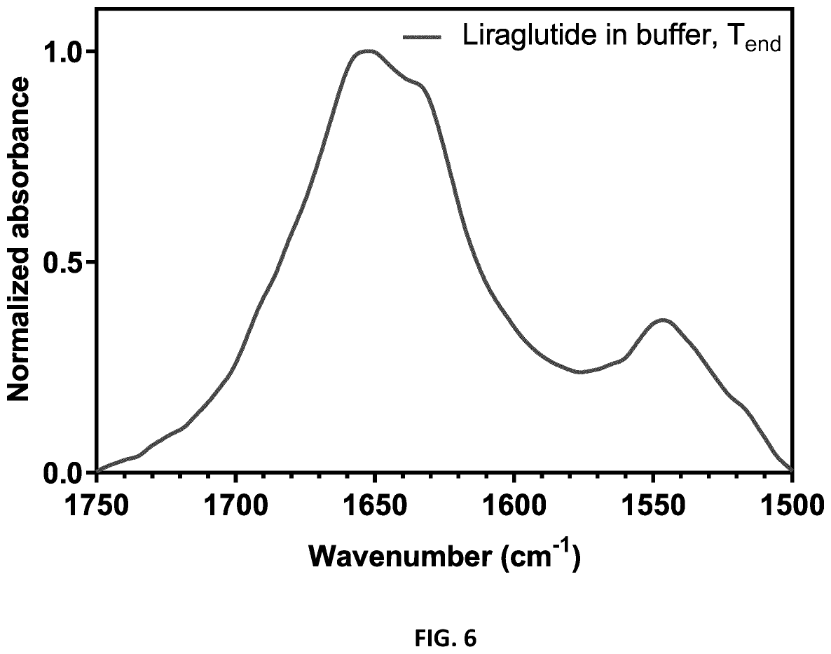

Interestingly, upon addition of cyclodextrin, full inhibition of the fibrillation was observed over the course of the assay. The absence of fibrils on the TEM pictures (FIG. 4) and the unchanged CD spectrum post-ThT assay (FIG. 3A) exclude the possibility of a false negative due to the presence of cyclodextrin and confirmed the inhibitory effect of the macrocyclic molecule. Interestingly, the ability to constrain fibril growth was also observed for liraglutide, another lipidated GLP1 analogue (FIGS. 5 and 6).

Finally, the co-formulation containing MEDI0382 and DPZ in HPβCD vehicle at pH 7 was also subjected to the ThT assay in order to assess the impact of the presence of DPZ on the peptide physical stability. Interestingly, DPZ did not hamper the inhibitory effect of the cyclodextrin, and no fibrillation occurred as confirmed by the TEM pictures (FIG. 4).

In order to determine the mechanism behind the aggregation inhibitory effect of HPβCD, a thorough characterization was performed to evaluate the interaction between the macrocycle and the actives molecules.

### Example 3: Cyclodextrin Forms an Inclusion Complex with Medi0382 and Dpz

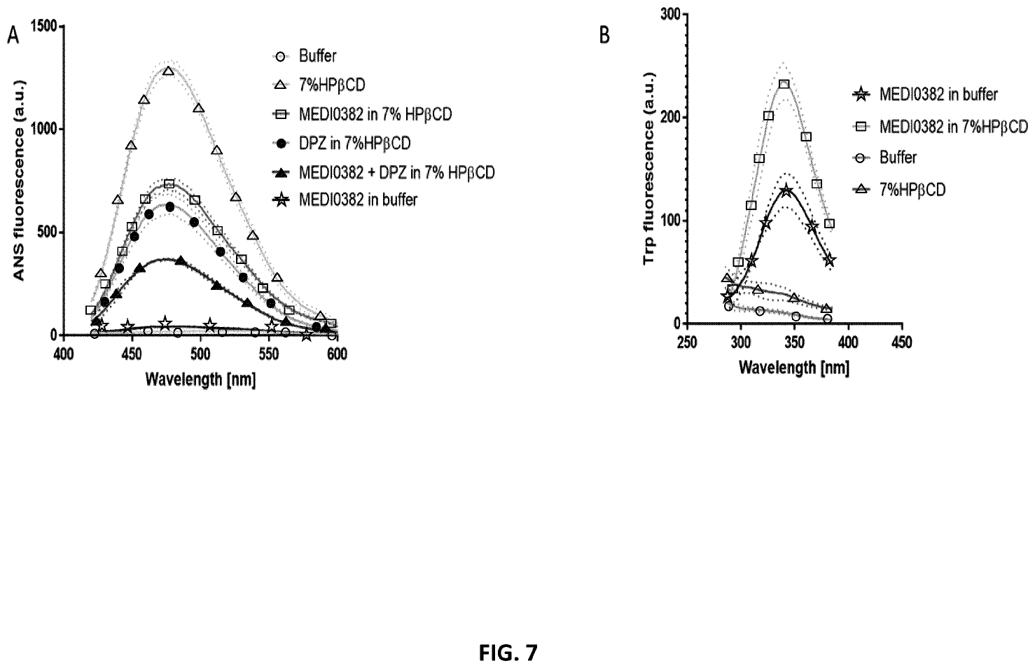

ANS is an amphiphilic dye that binds preferentially to hydrophobic cavities and whose fluorescence depends on its environment. In a polar environment, the fluorescence yield remains low, while an increase occurs upon interaction with hydrophobic surface. As ANS can form an inclusion complex with cyclodextrin (Nishijo J, et al., J Pharm Sci. 80(1):58-62 (1991)), it was used to qualitatively compare the hydrophobic core available in the various formulations (FIG. 7A). When added to the cyclodextrin vehicle, ANS fluorescence was greatly enhanced compared to that of the buffer due to the interaction with the cyclodextrin cavity. Interestingly, the fluorescence intensity was reduced when DPZ or MEDI0382 were formulated with the cyclodextrin, suggesting that both drug molecules formed an inclusion complex with the cyclodextrin, resulting in lower hydrophobic surface available for the ANS probe. The lowest intensity amongst the cyclodextrin formulations was observed for the co-formulation of DPZ and peptide which demonstrated that both molecules could interact with the cyclodextrin despite the presence of the other (FIG. 7A). ANS was also incubated with MEDI0382 in buffer, but the signal remained low as the peptide contains mainly alpha helix structure and therefore has low hydrophobic surface (FIG. 7A).

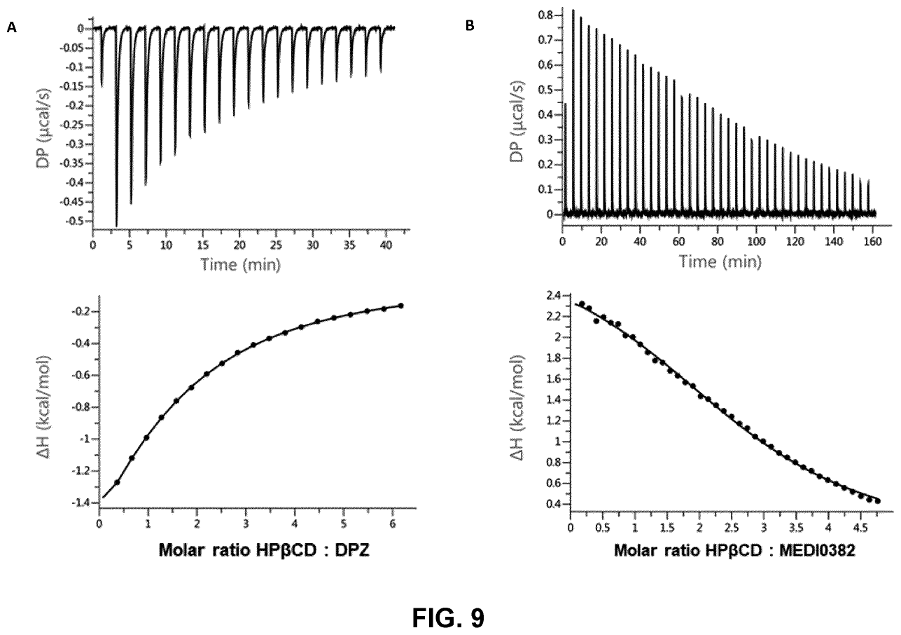

The complex with HPβCD was further characterised by isothermal titration microcalorimetry experiments (ITC) (FIG. 9). ITC is a label-free technique that permits determination of the thermodynamic parameters of biomolecular interactions by measuring the heat that is either released or absorbed during a binding event. (See Claveria-Gimeno R, et al, Expert Opin Drug Discov.12:363-77 (2017) and Klebe G., Nat Rev Drug Discov. 14:95-110 (2015)). In the past decade, ITC has increasingly become a technique of choice to characterize cyclodextrin-guest interaction due to its high sensitivity. This high sensitivity enables measuring dissociation constants from the millimolar to nanomolar range (Bouchemal K, et al., Drug Discov Today 17(11-12):623-9 (2012)). This approach was therefore used to study the interaction between MEDI0382 and HPβCD and compared to that of DPZ with HPβCD. Two opposite thermodynamics profiles were obtained from the titration of HPβCD into DPZ or peptide (FIG. 9). The binding of HPβCD:DPZ displayed an exothermic profile with equal contribution from the enthalpy and the entropy suggesting favourable hydrogen bonds and hydrophobic interaction. The affinity constant of the DPZ:HPβCD complex determined by ITC (6.6×10³ M⁻¹) was in good agreement with the value calculated from the phase solubility diagram (4.7×10³ M⁻¹). The 1:1 stoichiometry of HPβCD:DPZ previously determined through the phase solubility diagram was confirmed by ITC as shown in Table 2 below.

In contrast to DPZ, the interaction HPβCD:MEDI0382 appeared to be endothermic, characteristic of an entropy driven interaction dominated by hydrophobic interaction. The curve fitting from the ITC measurement suggested a stoichiometry 3:1. In comparison, a titration was also performed with glucagon, as well as with a non-lipidated analogue to MEDI0382. In both cases, no thermodynamic signal was observed, which suggests that the lipid chain is a key driver for the interaction between cyclodextrin and MEDI0382.

TABLE 2
ITC thermodynamic parameters of the interactions
HPβCD:MED10382 and HPβCD:DPZ

| | n | K (M⁻¹) | ΔH (kcal/mol) | −TΔS (kcal/mol) |
|---|---|---|---|---|
| HPβCD:MEDI0382 | 2.8 | 1.02E+04 | 2.9 | −8.4 |
| HPβCD:DPZ | 1.2 | 0.43E+04 | −4.2 | −0.8 |

### Example 4: HpβCd Forms a Complex with Medi0382 Through Interaction with the Aromatic Residues and the Lipid Chain

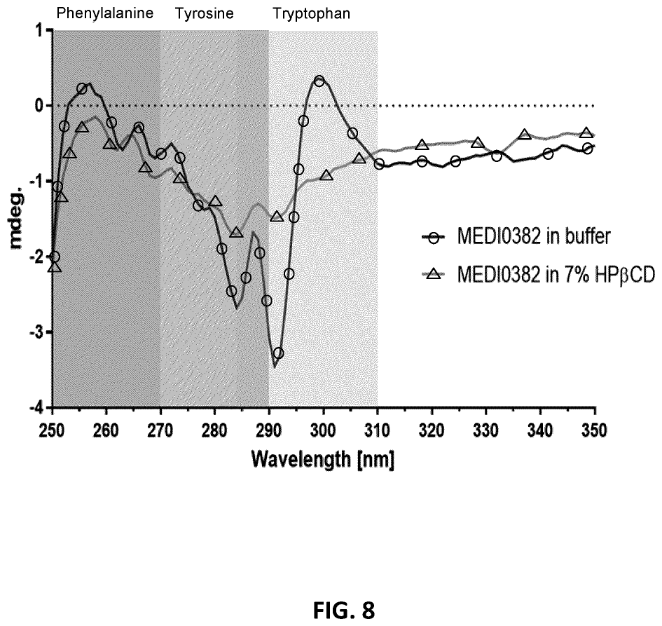

In order to gain some insight into the interaction between HPβCD and MEDIO382, a near UV CD analysis was performed on the formulation. As near UV CD is predominantly driven by the aromatic chromophores tyrosine (Tyr), phenylalanine (Phe), and tryptophan (Trp), a change in signal can provide information regarding their microenvironment. The spectra of MEDIO382 in buffer and MEDIO382 in cyclodextrin show different absorption patterns (FIG. 8) for each aromatic amino acid region, with Trp being the most affected. This suggests a change of local environment for all three chromophores either due to secondary structural change and/or direct interaction with HPβCD.

To further assess the interaction with Trp, intrinsic Trp fluorescence was monitored provide information regarding changes upon formulation in cyclodextrin (FIG. 7B). The measurements were performed with 0 and 7% cyclodextrin. The intrinsic tryptophan fluorescence maxima measured at 342 nm in buffer is indicative of a solvent-exposed Trp residue as previously reported for denatured proteins such as glucagon (352 nm) and melittin (346 nm) (Ghisaidoobe AB, et al., Int J Mol Sci. 15:22518-38 (2014)). Interestingly, when formulated in cyclodextrin, an approximately 2-fold increase of Trp fluorescence intensity was observed. The lower fluorescence intensity in buffer likely results from a quenching effect due to Trp exposure to water (Muino PL, et al., J Phys Chem B. 113:2572-7 (2009)), while the fluorescence increase could be associated with a transition toward a less polar environment such as the cavity of the HPB. Similar enhanced fluorescence emission was observed with free Trp molecule in presence of cyclodextrin. This observation indicates the existence of an interaction between HPβCD and the Trp residue of MEDIO382.

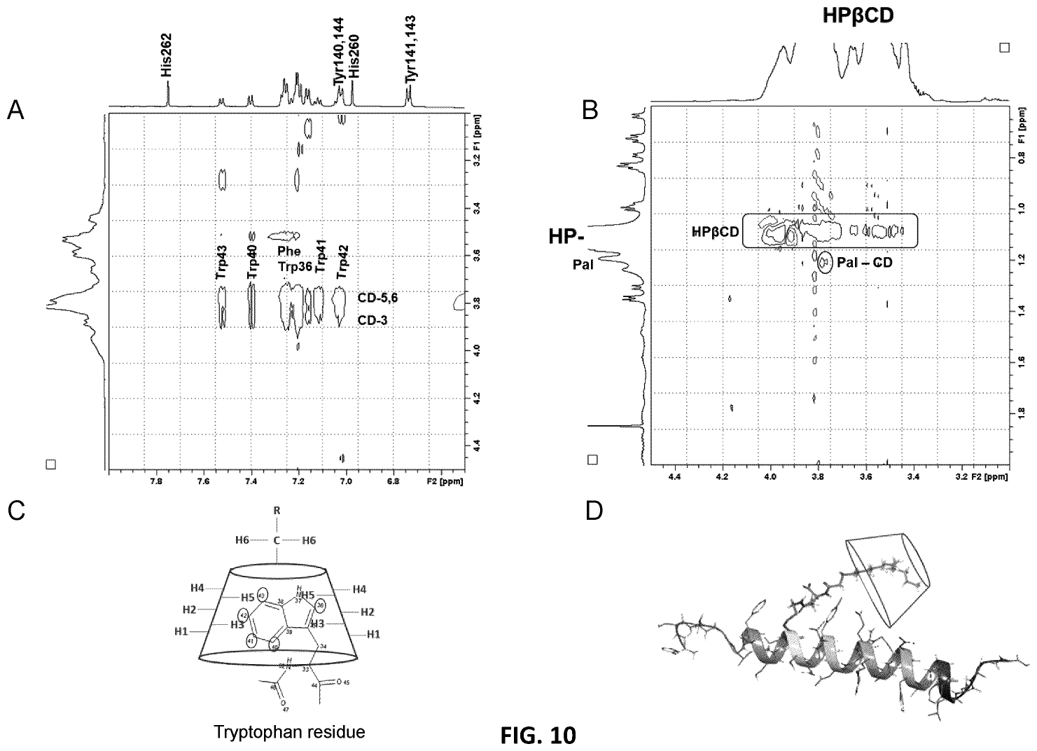

In order to confirm the interaction sites between cyclodextrin and the peptide, 2D NOESY NMR analysis was performed on the MEDI0382 cyclodextrin formulation compared to MEDI0382 in buffer. 2D NOESY NMR spectra were acquired with water suppression from solutions of MEDI0382 at 4.3 mg/ml in buffer with and without 10% HPβCD. The ratio of MEDI0382:cyclodextrin had to be reduced compared to the formulation so as to avoid a dynamic range problems in the NOESY NMR spectrum. Due to the insensitive nature of the NMR technique, the concentration of MEDI0382 was largely increased (˜10-fold higher than the formulation), whereas the cyclodextrin amount was only increased slightly to avoid the dynamic range problem and masking the peptide NMR signals. CD analysis confirmed that, despite the modification of the ratio, the secondary structure was equally affected with a conversion from alpha helix to β-sheet. The NMR spectrum of the peptide was previously authenticated. However all the amino acids have not yet been fully assigned in the current solution. The cyclodextrin resonance assignments were based on the literature values (Schneider et al. Chemical Reviews, 1998, Vol. 98, No. 5]. The spectra of MEDI0382 in the presence of HPβCD revealed strong interactions between the H-5 and H-6 protons of the HPβCD (FIG. 10B) and several protons from the peptide. Cross peaks at ˜7.55, 7.40, 7.1, 7.05 ppm in F2 and 3.80 ppm in F1 (FIG. 10A) indicates an NOE between H5/H-6 of HPβCD with the aromatic protons 43, 40, 41, 42 of Trp (FIG. 10B). Cross peaks at ˜7.25 ppm in F2 and 3.80 ppm in F1 (FIG. 10A) indicates an NOE between H5/H-6 of HPβCD and the aromatic proton 36 of Trp and the aromatic protons of Phe. Another NOE was observed at 3.75 ppm in F2 and 1.20 ppm in F1 (FIG. 10C), indicating an NOE between cyclodextrin and the lipid chain (FIG. 10D).

### Example 5: The Fibrillation Inhibitory Mechanism is Driven by Steric Hindrance Preventing Π-Π Stacking and Electrostatic Attraction and Lipid Interaction

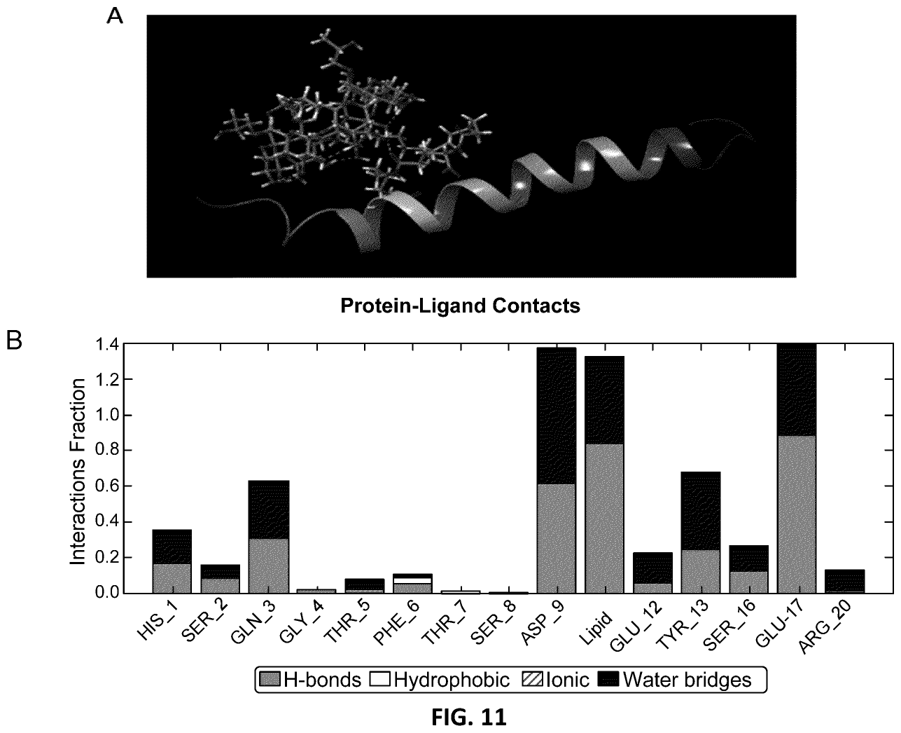

The stabilizing effect of cyclodextrin on peptide has previously been reported in the literature for insulin, amyloid-β, and glucagon (see Kitagawa K, et al., Amyloid. 22:181-6 (2015); Matilainen L, et al., J Pharm Sci. 97:2720-9 (2008); and Ren B, et al., Phys Chem Chem Phys.18:20476-85 (2016)). However, the effect was only evidenced through delay of lag-time of a few hours or reduction in fibril quantity; no full inhibition was achieved despite similar ratio peptide:cyclodextrin used in the case of insulin and glucagon. This difference is likely due to the fibrillation process of the peptide and the type of interactions involved between the cyclodextrin and the peptide. For peptides reported in the literature, Trp and Phe are common preferential interactions site with beta-cyclodextrins (see Kitagawa (2015); Matilainen (2008); Ren (2016); and Qin XR, et al., Biochem Biophys Res Commun. 297:1011-15 (2002)). The formation of an inclusion complex between cyclodextrin and the aromatic residues can possibly prevent inter/intramolecular Π-Π interactions. Interaction with Trp and Phe was clearly demonstrated for MEDI0382 through near UV, Trp fluorescence, and NMR analyses. Furthermore, a profound change of secondary structure was observed upon formulation in cyclodextrin with a decrease in alpha helix compensated by a high content of 3-sheet as estimated by CD pro. This conversion suggests that when formulated in cyclodextrin, the network of H-bond normally stabilising the helix structure is disrupted possibly due to preferential H-bondings between HPβCD and multiple amino acids. As the peptide NMR assignment has not yet been fully resolved, the analysis of the interaction was limited to the amino acids that have been assigned. Therefore a computational modelling was run to predict further interaction occurring between HPβCD and MEDIO382. Interestingly, the simulation showed the thermal motion of the lipid chain leading to the formation of an inclusion complex with the cavity of the cyclodextrin, which remained throughout the simulation. Moreover, the analysis of the simulation revealed numerous hydrogen bond interactions occurring between HPβCD and several amino acid residues including aspartic acid (Asp), glutamic acid (Glu), and the N-terminal histidine (His) (FIG. 11). Besides confirming the hypothesis of the secondary structure modification being driven by H-bonds, this observation gives a new insight about the fibrillation mechanism. The pKa of the acidic residues Glu and Asp as side chain are 3.9 and 4.0 respectively, while the N-terminal His presents two basic groups, an alpha-amino group and an imidazole group. In glucagon, which presents a similar structure to MEDIO382, the pKa of the 2 functional groups from His were reported to be 7.6 and 7.4 respectively (Hefford MA, et al., Biochemistry 24(4):867-74 (1985). Therefore, at pH 7, Glu and Asp are both negatively charged, whereas His is positively charged, which can contribute to fibril formation by electrostatic interaction. However, when MEDIO382 is formulated in cyclodextrin, the inclusion complex with the charged residue could sterically prevent self-assembly. Lastly, while the role of the lipid chain in the aggregation process is not well understood, the interaction with the cyclodextrin confirmed by NMR, ITC and simulation can generate further steric hindrance to self-assembly.

### Example 6: Effect of pH on Incretin Peptides Co-Formulated with Dpz in Cyclodextrin

In order to evaluate the effect of pH on incretin peptides with DPZ in cyclodextrin, co-formulations were evaluated at pH 6.5 and 8 using (i) intrinsic trp fluorescence, (ii) circular dichroism (CD) pre- and post-Tht assay, and (iii) TEM and atomic force microscope (AFM) picture post Tht assay.

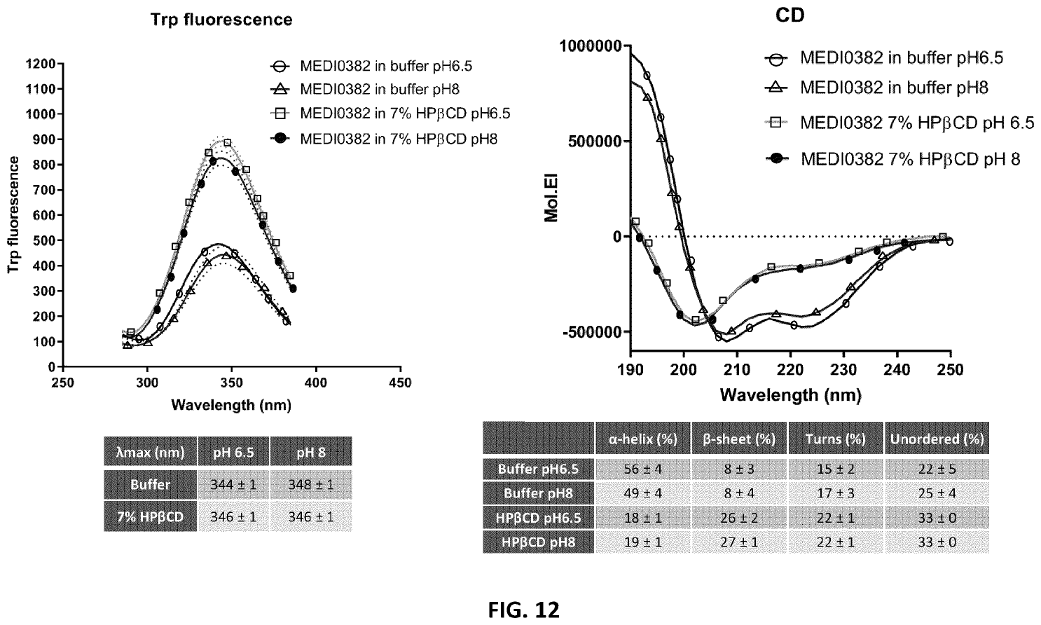

Intrinsic Trp fluorescence was used to compare formulations of MEDI0382 at pH 6.5 and 8 in the presence and absence of cyclodextrin. In the absence of cyclodextrin, the pH increase from 6.5 to 8 was associated with a trp fluorescence red shift from 344 nm to 348 nm respectively. In contrast, when formulated in cyclodextrin, trp λmax was measured at 346 nm regardless of the pH. In addition, the fluorescence intensity underwent a 2-fold increase (FIG. 12, left). Interestingly, the far UV CD spectra also showed a dramatic structural modification when formulated in cyclodextrin with a loss of helicity down to 18-19% compensated by an increase in j-sheet and random coil (FIG. 12, right).

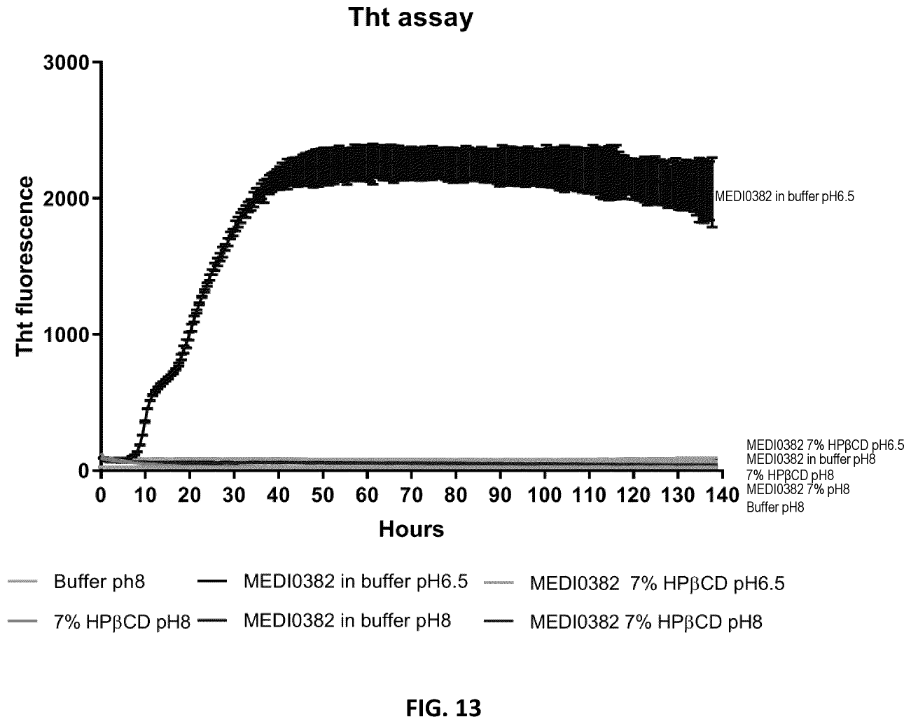

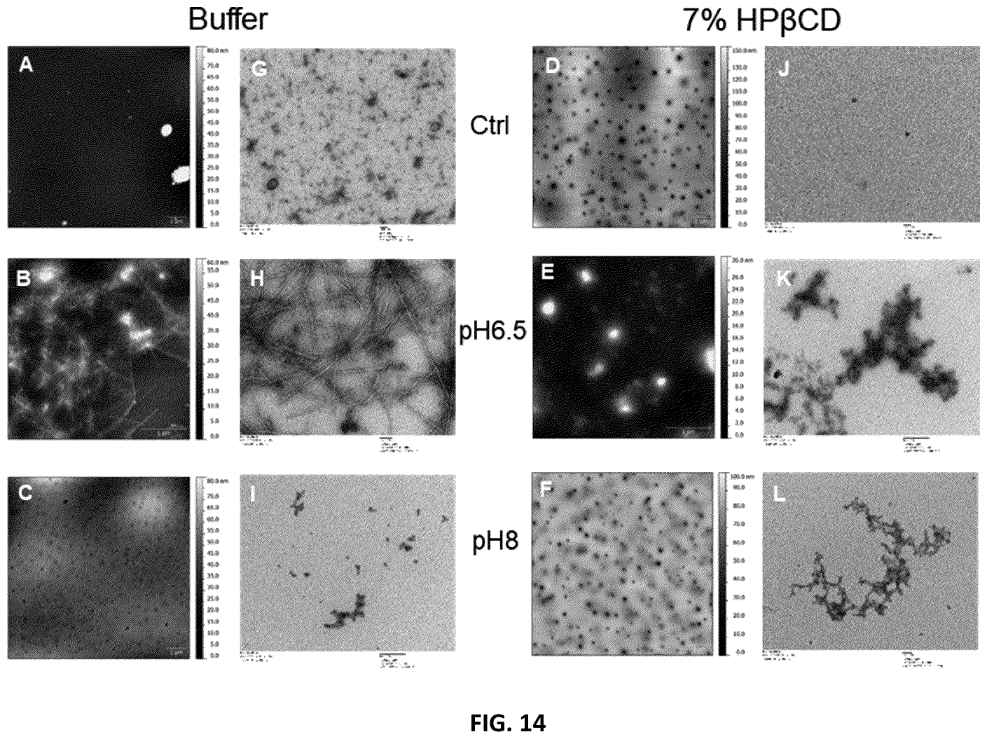

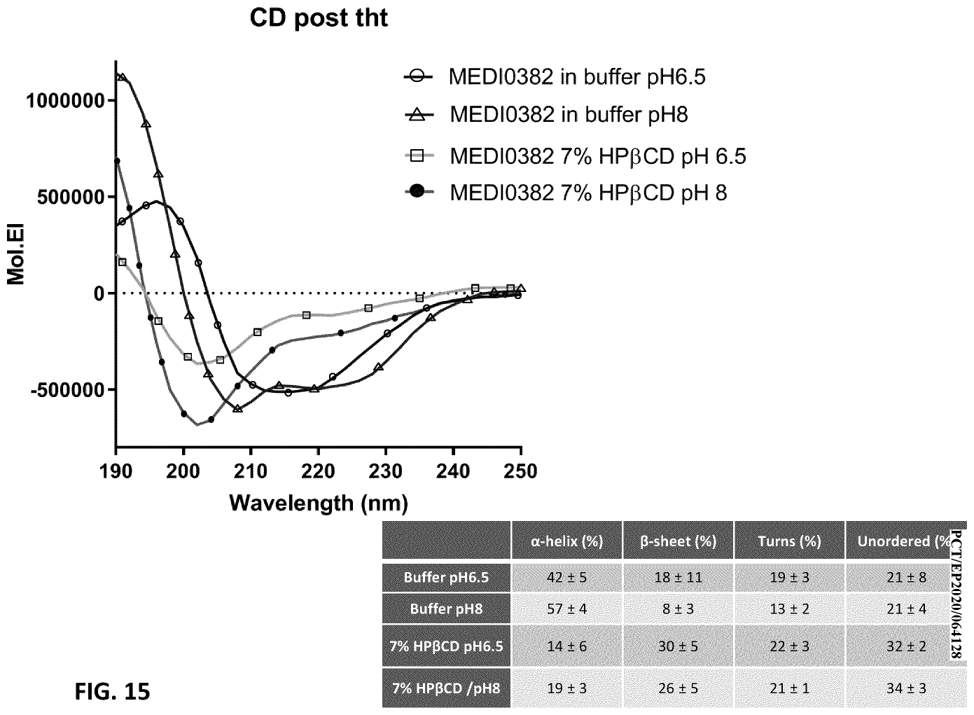

At pH 6.5, the Tht profile showed a very short lag time followed by a 30 hour growth phase before reaching a plateau (FIG. 13). The AFM picture taken at the end of the assay confirmed the presence of fibers (FIG. 14), and the far UV CD spectrum post Tht assay indicated a loss of helicity (FIG. 15). When increasing the pH to 8, the Tht fluorescence remained at the level of the buffer control (FIG. 13) suggesting no fiber formation as verified by the absence of ordered aggregates on the AFM pictures (FIG. 14).

Interestingly, upon addition of cyclodextrin at pH 6.5, while fibrilisation of MEDI0382 in buffer occurred rapidly (lag time=3 hours), the cyclodextrin fully inhibited the aggregation over the course of the assay (FIG. 13). The absence of fibers on the AFM picture (FIG. 14) and the unchanged CD spectrum pre- and post-Tht assay (FIG. 15) exclude the possibility of a false negative due to the presence of cyclodextrin and confirmed the inhibitory effect of the macrocyclic molecule.

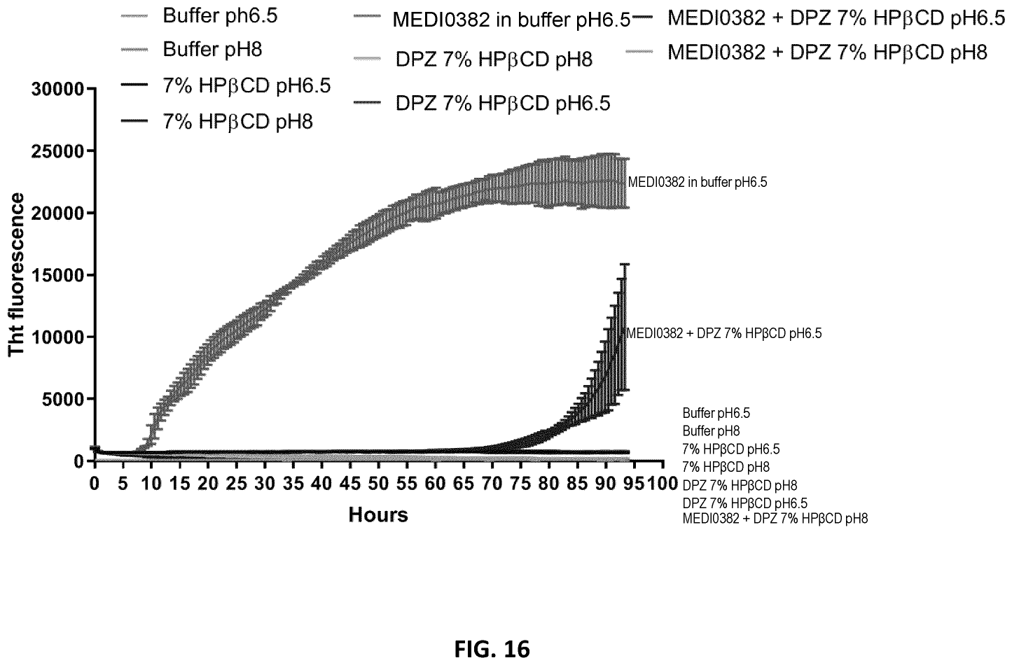

An aggregation kinetic assay was also performed with the co-formulation at ph 6.5 and pH 8. The Tht assay at pH6.5 showed an increased in fluorescence at 75 hours for the co-formulation, which was not seen for MEDI0382 alone in cyclodextrin (FIG. 16). However in comparison to MEDI0382 in buffer, the lag phase was much longer for the co-formulation meaning that the fibrillation was still delayed (FIG. 16). Contrary to pH 6.5, at pH 8 the co-formulation proved to be stable (FIG. 16).

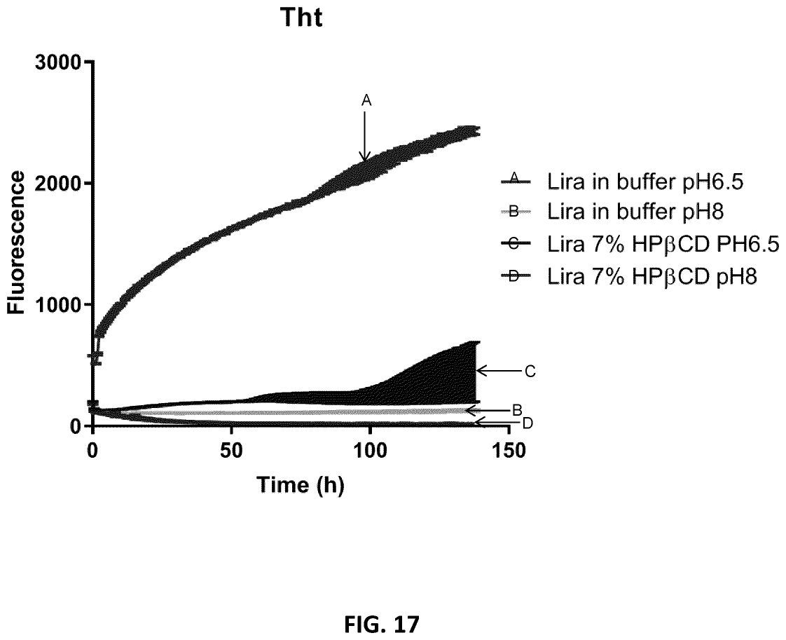

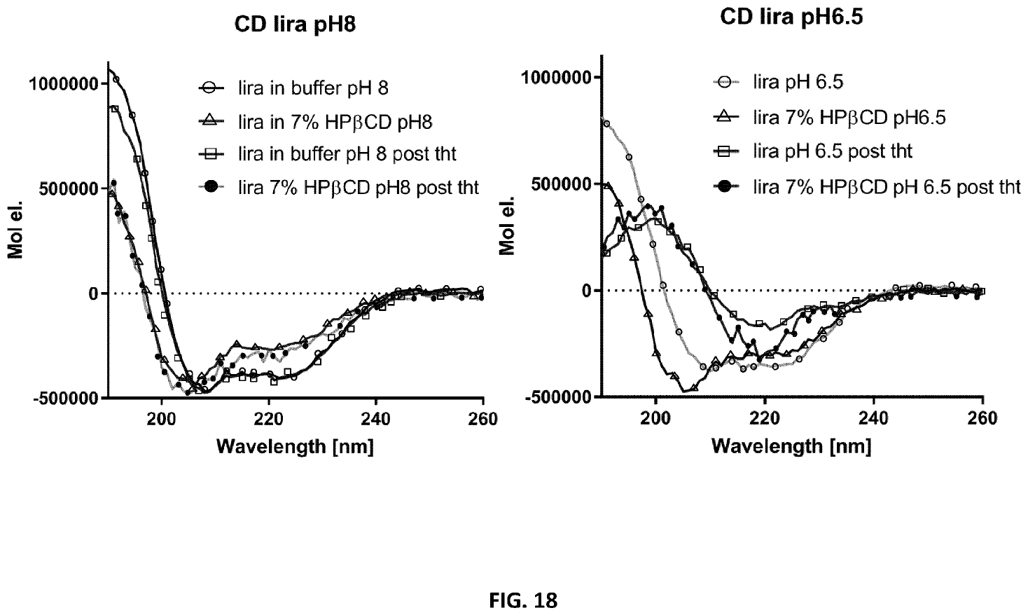

Similar experiments were also performed using liraglutide co-formulations. Cyclodextrin seems to reduce liragludite fibrillation at pH 6.5 as measured in a Tht assay (FIG. 17). Similarly to MEDIO382, cyclodextrin changes the secondary structure of liraglutide at both pH 6.5 and pH 8, and the CD at pH 6.5 post-Tht confirmed the presence of fibers in buffer and cyclodextrin formulation (FIG. 18).

These results demonstrate that cyclodextrin can increase the stability of lipidated incretin peptides at least from pH 6.5 to 8.

### Example 7: Medi0382 Co-Formulated with Dpz in Cyclodextrin Maintains Potency

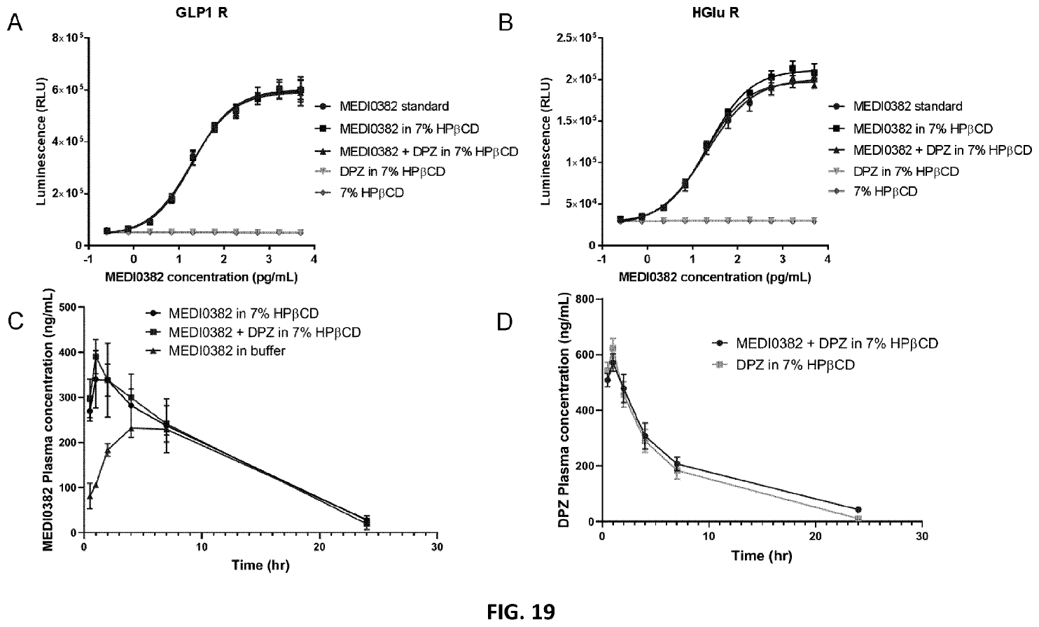

Although HPβCD enhances the physical stability of the peptide, the loss of alpha-helix could have a dramatic impact on the potency of the peptide. Several studies have indeed evidenced that the secondary structure of GLP-1 and GLP-1 analogue plays a fundamental role in the binding to and activation of the corresponding receptor (see e.g., Donnelly D., Br J Pharmacol. 166:27-41 (2012)). More especially, the α-helical structure appears to be a key factor driving the affinity and potency of the peptide (Adelhorst K, et al., J Biol Chem. 269:6275-8 (1994)). The biological activity of MEDI0382 was therefore evaluated in vitro on GLP1 and Glucagon receptors to assess the impact of the presence of cyclodextrin and DPZ on its agonist properties. The in vitro potency was assessed on CHO cells over-expressing human recombinant GLP-1 or glucagon receptors, and the activity was reported as EC50 values after measurement of cAMP accumulation. As shown in FIGS. 19A and 19B, no change of EC50 for either receptor was observed regardless of the formulation. Additionally, it is worth noting that neither the vehicle nor DPZ in vehicle showed activity on the receptors. The co-formulation is compatible with once-daily dose frequency for both MEDI0382 and DPZ.

The performance of MEDI0382 in the co-formulation was finally evaluated in vivo to assess the impact of the cyclodextrin and the presence of DPZ (FIGS. 19C and D). The pharmacokinetic study of MEDI0382 was performed in cyclodextrin alone or co-formulated with DPZ and compared to that of MEDI0382 in buffer following subcutaneous injection in rats. The plasma concentration vs time profile and associated PK parameters of MEDI0382 are reported in FIG. 19C and Table 3. Plasma concentration time profile of DPZ are presented in FIG. 19D.

TABLE 3
Mean PK parameters for MEDI0382

| MEDI0382 | | Cmax | | Half-life | AUC_{inf} |
|---|---|---|---|---|---|
| Dose | | (SD) | Tmax ¹ | (SD) | (SD) (ng · |
| (mg/Kg) | Formulation | (ng/mL) | (hr) | (hr) | hr/mL) |
| 0.05 | Buffer | 235 | 4 | 5.7 | 3207 |
| | | (5.4) | | (0.3) | (237) |
| 0.05 | MEDI0382 in | 346 | 1 | 5.8 | 3850 |
| | 7% HPβCD | (74) | | (0.5) | (972) |
| 0.05 | MEDI0382 + | 391 | 1 | 4.9 | 3682 |
| | DPZ in 7% | (37) | | (1.2) | (531) |
| | HPβCD | | | | |

Cmax = maximal plasma concentration; Tmax = time of maximal plasma concentration; AUC_{inf} = Area under the plasma concentration time curve to infinite; SD = standard deviation
¹ Tmax is reported as median values

MEDI0382 in buffer showed slow absorption kinetics, with the maximal concentration reached at 4 hr post injection. When formulated alone in cyclodextrin, the Tmax was significantly shortened (1 hr vs 4 hr for the HPB and buffer formulations, respectively), and the Cmax was approximately 1.5-fold higher than the buffer formulation. Similarly, the overall exposure was increased 1.2-fold in the presence of cyclodextrin The presence of DPZ in the co-formulation group did not induce any further change to the PK compared to MEDI0382 in cyclodextrin. The elimination phase of MEDIO382 remained similar in all three formulations suggesting that the formulation does not impact elimination of MEDI0382 mainly driven by the binding to albumin.

Additionally, the PK of DPZ was unchanged in the presence of MEDIO382.

These data demonstrate that co-formulations of MEDI0382 and DPZ in cyclodextrin maintain the stability and biological potency of MEDI0382 on both GLP1 and Glucagon receptors in vitro and in vivo. Thus, the co-formulations are compatible with once-daily dose frequency for both MEDT0382 and DPZ.

What is claimed is:

1. A liquid pharmaceutical composition comprising (i) a lipidated incretin peptide, (ii) a sodium glucose co-transporter 2 inhibitor (SGLT2i), and (iii) a cyclodextrin.

2. The composition of claim 1, wherein the incretin peptide is monolipidated.

3. The composition of claim 1 or 2, wherein the incretin peptide is a GLP-1/glucagon dual agonist peptide.

4. The composition of any one of claims 1-3, wherein the incretin peptide is MEDIO382, liraglutide, or semaglutide.

5. The composition of any one of claims 1-4, wherein the SGLT2i is dapagliflozin.

6. The composition of any one of claims 1-5, wherein the cyclodextrin is a beta cyclodextrin, optionally wherein the beta cyclodextrin is hydroxypropyl-β-cyclodextrin.

7. The composition of any one of claims 1-5, wherein the cyclodextrin is sulfobutyl ether cyclodextrin.

8. The composition of any one of claims 1-7, wherein the lipidated incretin peptide is present at a concentration of about 0.5 mg/mL.

9. The composition of any one of claims 1-8, wherein the SGLT2i is present in a concentration of about 17 mg/ml.

10. The composition of any one of claims 1-9, wherein the cyclodextrin is present at a concentration of about 7% w/v.

11. The composition of any one of claims 1-10, wherein the SGLT2i and the cyclodextrin have a stoichiometry of about 1:1.

12. The composition of any one of claims 1-11, wherein the composition has a pH of about 6.5 to about 8 or about 7 to about 8, optionally wherein the composition has a pH of about 7.

13. The composition of any one of claims 1-12, wherein the composition has a volume of 1 mL or less.

14. The composition of any one of claims 1-13, wherein the composition is for parenteral administration, optionally wherein the parenteral administration is subcutaneous administration.

15. The composition of any one of claims 1-14, wherein the composition comprises inclusion complexes comprising the lipidated incretin peptide, the SGLT2i, and the cyclodextrin.

16. The composition of any one of claims 1-15, wherein the composition does not contain fibrils of the lipidated incretin peptide.

17. The composition of any one of claims 1-16, wherein the composition does not decrease the affinity of the lipidated incretin peptide for the GLP-1 receptor and/or the glucagon receptor.

18. The composition of any one of claims 1-17, wherein administration of the composition to a rat produces a lipidated incretin peptide Cmax of about 390 ng/ml, a lipidated incretin peptide T max of about 1 hour, a lipidated incretin peptide half-life of about 5 hours, and/or a lipidated incretin peptide AUC_{0-inf} of about 3500-4000 ng.hr/mL.

19. An injection pen comprising the composition of any one of claims 1-18, optionally wherein the injection pen delivers about 600 μL of the composition.

20. A method of treating type 2 diabetes in a subject in need thereof comprising administering the composition of any one of claims 1-18 to the subject, optionally wherein the subject is overweight or obese.

21. A method of treating Nonalcoholic Steatohepatitis (NASH) or Nonalcoholic Fatty Liver Disease (NAFLD) in a subject in need thereof comprising administering the composition of any one of claims 1-18 to the subject, optionally wherein the subject is overweight or obese.

22. A method of reducing liver fat in a subject in need thereof comprising administering the composition of any one of claims 1-18 to the subject, optionally wherein the subject is overweight or obese.

23. The method of any one of claims 20-22, wherein the administration delivers about 10 mg of the SGLT2i and/or about 300 μg of lipidated incretin peptide to the patient.

24. The method of any one of claims 20-23, wherein the administration is an adjunct to diet and exercise.
